SEQUENCE LISTING (26 sequences)

SEQ ID NO: 1 - protein, 589 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARVGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGFLALASDLKERGSDGDGHWRRSIPAFHI

SEQ ID NO: 2 - DNA, 3585 bp, Homo sapiens
  CDS (99)...(1865)
cacggacgcgtgggcgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggttggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggtttcctagctcttgcctcagaccttaaagagagagggtctgatggggatgggcactggagacggagcatcccagcatttcacatctgagctggctttcctctgccccaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaaaaaaaaaaaaaaaaaaaaaaagggcggccgc

SEQ ID NO: 3 - protein, 746 aa, Rattus norvegicus
MWTRSLPLGSRSLSDRDLRTEPVLGSPRDITACSPRAQCPAFTSFPRPRAPLLAPMGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 4 - DNA, 3372 bp, Rattus norvegicus
  CDS (40)...(2277)
ggcgtccagcacccacaccctaaaaggctttccatctttatgtggacgcgcagtctgccactgggctcccgttctctctctgaccgagacctgaggactgagccagtgcttggctcccctagggacatcacagcctgcagtcccagagcccagtgccccgcgttcacgtccttcccgaggcctcgtgcacctctcctcgccccgatgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagccatggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacgtcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccagtgacagcctcaggcaggcattgtacctgtggctggacgcagagatggacgtcctggctctcttgtgtctagccaaaagtggggagactctgcctgggggaacttggcgtctcatcctgggtacccattcctggtgtatgtgtggggaagcacctccttcatggtcagggggcctgtgcttggccttctgccatcgaagatgttaagctatagttggctttggccagctgctccagtatatcagaacctgagagcacttgctacaaggctagtgttcaggccttaggcctccagagtgaatgtatcctgcaggaagataatgatggatcgtgacccttgacggtcacccccctcccccaggtcagatgtcaccagactagaacagtatctgaaagctgctggggccactcacagcttgcttactctggagacagcattttgggctccctgattaatgcagatcagttctgcccacctccaggggtggatccagctgtgaggctcacctgtatcttccagatgttctcatctgctgcaccgaaggctctggccctgctcaggagaacacgctacgaactcctagctgactctgtttgcactggagaaccacacagggcttaccccactaccctgtgcactgactggcttcactttatgaaggaagagacagggccagagaagcaatgtcatgcagccagtgatgctaggacataaatccagagtggctggccctgaagccatgcctcttggcaatgccaggctgggcatcctatttttgaagcaaacaaaaaatgagaggacaggctgtgcttcagcggcttgttcctggacctatgctcccttagccccagtcccacggattatgtggagagtggaggagcaacagagggcgactgtactaaggccacacaagtcgacaagaacacctatatccttttgacctcttctgcttttttatagtaagctttccctacctgcgttgcttttgtaactcgatatttatgccgggttttatagagtttttattatgtagtgacttttcagaataaaagaagctgatgtgactgcctgaaaaaaaaaaaaaaagggcggccgc

SEQ ID NO: 5 - protein, 704 aa, Mus musculus
MSFPRPRAPLLAPMGTHCSAWLRWPLLPLLLLLLLLLCPTGAGTQDEDGDYEELMLALPSQEDGLADEAAHVATATFRRCSKEAWRLPGTYIVVLMEETQRLQIEQTAHRLQTRAARRGYVIKVLHIFYDLFPGFLVKMSSDLLGLALKLPHVEYIEEDSFVFAQSIPWNLERIIPAWHQTEEDRSPDGSSQVEVYLLGTSIQGAYREIAGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNAACQHLARTGVVLVAAAGNFRDDACLYSPASAPEVITVGATNAQDQPVTLGTLGTNFGRCVDLFAPGKDIIGASSDCSTCFMSQSGTSQAAAHVAGIVARMLSREPTLTLAELRQRLIHFSTKDVINMAWFPEDQQVLTPNLVATLPPSTHETGGQLLCRTVWSAHSGPTRTATATARCAPEEELLSCSSFSRSGRRRGDWIEAIGGQQVCKALNAFGGEGVYAVARCCLVPHANCSIHNTPAARAGLETHVHCHQKDHVLTGCSFHWEVEDLSVRRQPALRSRRQPGQCVGHQAASVYASCCHAPGLECKIKEHGISGPSEQVAVACEAGWTLTGCNVLPGASLTLGAYSVDNLCVARVHDTARADRTSGEATVAAAICCRSRPSAKASWVQ

SEQ ID NO: 6 - DNA, 2259 bp, Mus musculus
  CDS (145)...(2256)
ggaccttcacgtggacgcgcaggctgccggtgggctcccgttctctctctctttctgaggctagaggactgagccagtccttggctccccagagacatcacggcccgcagccccggagccaagtgccccgagtcccaggcgtccatgtccttcccgaggccgcgcgcacctctcctcgccccgatgggcacccactgctctgcgtggctgcggtggccgctgttgccgctgctgctgctgctgttgctgctactgtgccccacgggcgctggtacccaggacgaggacggagattatgaagagctgatgctcgccctcccgtcccaggaggatggcctggctgatgaggccgcacatgtggccaccgccaccttccgccgttgctccaaggaggcctggaggctgccaggaacctacattgtggtgctgatggaggagacccagaggctacagattgaacaaactgcccaccgcctgcagacccgggctgcccgccggggctatgtcatcaaggttctacatatcttttatgacctcttccctggcttcttggtgaagatgagcagtgacctgttgggcctggccctgaagttgccccatgtggagtacattgaggaagactcctttgtcttcgcccagagcatcccatggaacctggagcgaattatcccagcatggcaccagacagaggaagaccgctcccctgatggaagcagccaggtggaggtgtatctcttaggtaccagcatccagggtgcctatcgggagattgcgggcagggtcaccatcaccgacttcaacagtgtgccggaggaggatgggacacgcttccacagacaggcgagcaagtgtgacagccacggcacccacctggcaggtgtggtcagcggccgggatgctggtgtggccaagggcaccagcctgcacagcctgcgtgtgctcaactgtcaagggaagggcacagtcagcggcaccctcataggcctggagtttattcggaagagtcagctaatccagccctcggggccactcgtggttctgctgcccctggccggtgggtatagccgcatcctcaacgctgcctgccagcacctggcgaggactggggtggtgctggttgcagcagctgggaacttccgggacgacgcctgcctctactccccagcttctgctccagaggtcatcacagtcggggccacgaatgcccaggaccagccagttaccttggggactttggggactaattttggacgctgtgtggatctctttgcccccgggaaggacatcatcggagcgtccagtgactgcagcacatgcttcatgtcacagagtgggacctcacaggctgctgcccacgtggccggcattgtggctcggatgctgagccgggagcccacacttaccctggccgagctgcggcagaggctgatccacttctctaccaaagacgtcatcaacatggcctggttccctgaggaccagcaggtgctgacccccaacctggtggccacactgccccccagcacccatgagacaggcgggcagctgctctgtaggacggtgtggtcggcgcactcggggcccactcggacagctacagctacagcccgctgtgccccagaagaggagctgctgagctgctccagcttctccaggagcgggaggcgtcgtggtgattggattgaggccataggaggccagcaggtctgcaaggccctcaatgcatttgggggtgagggtgtctatgccgtcgcgagatgctgcctggttccccatgccaactgcagcatccacaacacccctgcagccagagctggcctggagacccatgtccactgccaccagaaggaccatgttctcacaggctgcagcttccattgggaagtggaagaccttagtgtccggaggcagcctgcgctgaggtccagacgtcagcctggccagtgcgttggccaccaggcggccagtgtctatgcttcctgctgccatgccccagggctggaatgcaaaatcaaggagcatgggatctcaggtccttcagagcaggtcgctgtggcctgtgaagcaggatggaccctgactggatgcaatgtgctccctggggcatccctcactctgggagcctacagcgtggacaacctgtgtgtggcaagagtccatgacactgccagagcagacaggaccagtggagaagccacagtagctgctgccatctgctgccggagccggccttcagcaaaggcctcctgggttcagtga

SEQ ID NO: 7 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 8 - DNA, 3617 bp, Homo sapiens
  CDS (245)...(2320)
cccacgcgtccggcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaaaaaaaaaaaaaaaaawaaaaaaaaaa

SEQ ID NO: 9 - protein, 691 aa, rattus norvegicus
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 10 - DNA, 2073 bp, rattus norvegicus
  CDS (1)...(2073)
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagccatggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacgtcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccag

SEQ ID NO: 11 - protein, 691 aa, Artificial Sequence
  mutant of rat sequence mutating amino acid His225Trp of SEQ ID NO9
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSWGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 12 - DNA, 2073 bp, Artificial Sequence
  mutant of rat sequence mutating nucleotides 673-675 CAT to TGG
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagctggggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacgtcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccag

SEQ ID NO: 13 - protein, 691 aa, Artificial Sequence
  mutant of rat sequence mutating amino acid Ser385Ala of SEQ ID NO9
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTAQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 14 - DNA, 2073 bp, Artificial Sequence
  mutant of rat sequence mutating nucleotides1153-1155 TCA to GCA of SEQ ID NO10
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagccatggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacggcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccag

SEQ ID NO: 15 - protein, 691 aa, Artificial Sequence
  mutant of rat sequence mutating amino acid His225Trp and Ser385Ala of SEQ ID NO9
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSWGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTAQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 16 - DNA, 2073 bp, Artificial Sequence
  mutant of rat sequence mutating nucleotides 673-675 CAT to TGG and nucleotides 1153-1155 TCA to GCA of SEQ ID NO10
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagctggggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacggcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccag

SEQ ID NO: 17 - protein, 425 aa, Artificial Sequence
  mutant of rat sequence truncated sequence ends at amino acid Met 425
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINM

SEQ ID NO: 18 - DNA, 1275 bp, Artificial Sequence
  mutant of rat sequence truncated sequence ends at nucleotide 1275
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagccatggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacgtcacaagctgctgcccacgtggctggcattgtggctatgatgctgaaccgggatccagcacttaccctggctgagctgcggcagaggttgatcctcttctctaccaaagatgtcatcaacatg

SEQ ID NO: 19 - protein, 453 aa, Artificial Sequence
  mutant of rat sequence truncated sequence ends at amino acid Gln453
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQ

SEQ ID NO: 20 - DNA, 1359 bp, Artificial Sequence
  mutant of rat sequence truncated sequence ends at nucleotide 1359
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagccatggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacgtcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcag

SEQ ID NO: 21 - protein, 507 aa, Artificial Sequence
  mutant of rat sequence truncated mutant ends at amino acid Val507
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQV

SEQ ID NO: 22 - DNA, 1521 bp, Artificial Sequence
  mutant of rat sequence truncated mutant ends at nucleotide 1521
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagacaggcgagcaagtgtgacagccatggcacccacctagcaggtgtggtcagcggccgggatgctggtgtggccaagggcaccagtctgcacagtctgcgtgtgctcaactgtcaagggaagggcacagtcagcggcaccctcataggcctggagtttattcggaagagccagctaatccagccttcggggccactcgtggtgctgctgcccctggcgggtgggtatagccggatccttaacactgcctgccagcgcctggcaaggactggggtagtgctggtggcagcagctgggaatttccgagatgatgcctgcctctactccccagcctctgctccagaggtcattacagttggggccactaatgcccaggaccagccagtcaccctggggactttggggacaaactttggacgctgtgtggatctctttgcccccgggaaggacatcatcggagcctccagtgactgtagcacgtgctacatgtcacagagtgggacgtcacaagctgctgcccacgtggctggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtc

SEQ ID NO: 23 - protein, 412 aa, Artificial Sequence
  mutant of rat sequence deletion mutant removes amino acids Leu147-Met425, inclusive, of SEQ ID NO9
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 24 - DNA, 1236 bp, Artificial Sequence
  mutant of rat sequence deletion mutant removes nucleotides 439-1275, inclusive, of SEQ ID NO10
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcagcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccag

SEQ ID NO: 25 - protein, 516 aa, Artificial Sequence
  mutant of rat sequence deletion mutant removes Gln218-Ala392, inclusive, of SEQ ID NO9
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 26 - DNA, 1548 bp, Artificial Sequence
  mutant of rat sequence deletion mutant removes nucleotides 652-1176, inclusive, of SEQ ID NO10
atgggcatccgctgctctacatggttgcggtggccgctgtcgccgcagctgctgttgttgctgctactgtgccccacaggctcccgtgcccaggacgaggacggagactacgaagagctgatgctcgccctcccgtcccaggaggatagcctggttgatgaggcctcacacgtggccaccgccaccttccgccgttgctccaaggaggcctggaggctgccaggaacctacgtggtggtgctgatggaggagacccagcggctgcaggttgaacaaactgcccatcgcctgcagacctgggcggcccgccggggctatgtcatcaaggttctgcatgtcttttatgacctcttccctggcttcttggtgaagatgagcagtgacctgttgggcctggccctgaagttgccccatgtggagtacatcgaggaagactcattagtcttcgcccagagcatcccatggaacctggagcggattatcccagcgtggcagcagacagaggaagatagctcccctgacggaagtagccaggtggaggtgtatctcttagataccagcatccagagtggccaccgggagatcgagggcagagttaccatcactgacttcaacagtgtgcctgaggaggacgggacacgtttccacagaggcattgtggctatgatgctgaaccgggatccagcacttaccctggctgagctgcggcagaggttgatcctcttctctaccaaagatgtcatcaacatggcctggttccctgaggaccagcgggtgctgacccccaaccgggtggccacactgccccccagcacccaggagacaggcgggcagctgctctgccggacagtgtggtccgcccactcagggcccacccgtacagcaacagccacagcccgctgtgcccctgaagaggaactgctgagctgctccagcttctccaggagcgggaggcgacggggtgatcgaattgaggccatagggggccagcaggtctgcaaggccctcaatgcatttgggggtgagggtgtctatgctgtcgcaaggtgctgcctgcttccccgtgtcaactgcagcatccacaacactcctgcagccagagctggtccgcagacccccgtccactgccaccagaaggaccatgttctcacaggctgcagcttccactgggaagtggaaaaccttagagcccagcagcagcctctgctgaggtccagacatcaacctggccaatgcgttggccaccaggaggccagtgtccacgcttcctgctgccatgctccaggtctggaatgcaaaatcaaggagcatggcatcgcaggtcctgcagagcaggtcaccgtggcctgtgaggcaggatggaccctgactggatgcaacgttctccctggggcatccctccctctgggggcctacagtgtggacaacgtgtgtgtggcacgaatccgtgatgctggtagagcggacaggaccagtgaagaagccacggtagctgctgccatctgctgccggagccggccttcggcaaaggcctcctgggttcaccag

(19) United States
(12) Patent Application Publication
(10) Pub. No.: US 2003/0119038 A1
(43) Pub. Date: Jun. 26, 2003

(54) NARC1, novel subtilase-like homologs

(76) Inventors: Brendan William Bingham, Newtown, PA (US); Lillian Wei-Ming Chiang, Princeton, NJ (US); Lorayne P. Jenkins, Hightstown, NJ (US); Ching-Hsiung Frederick Lo, Pennington, NJ (US); Saule Naureckiene, Old Bridge, NJ (US); Bradley Alton Ozenberger, Newtown, PA (US); Andrew Wood, Newtown, PA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/287,290

(22) Filed: Nov. 1, 2002

Related U.S. Application Data
(63) Continuation-in-part of application No. 09/517,906, filed on Mar. 3, 2000.
Application No. 09/517,906 is a continuation-in-part of application No. 09/499,235, filed on Feb. 7, 2000.
Application No. 09/499,235 is a continuation-in-part of application No. 09/393,174, filed on Sep. 9, 1999, now abandoned.
(60) Provisional application No. 60/161,188, filed on Oct. 22, 1999.

(51) Int. Cl. C12Q 1/68; G01N 33/574; C07H 21/04; C12N 9/99; C12P 21/02; C12N 5/06

(52) U.S. Cl. 435/6; 435/7.23; 435/69.2; 435/184; 435/320.1; 435/325; 536/23.2

(57) ABSTRACT

The present invention relates to a newly identified human and mouse programmed cell death (PCD) protein having homology to mammalian subtilases. The invention also relates to polynucleotides encoding the protein. The invention further relates to methods using the polypeptides and polynucleotides as a target for diagnosis and treatment in disorders mediated by or related to the protein. The invention further relates to drug-screening methods using the polypeptides and polynucleotides to identify agonists and antagonists for diagnosis and treatment. The invention further encompasses agonists and antagonists based on the polypeptides and polynucleotides. The invention further relates to procedures for producing the polypeptides and polynucleotides.

### CROSS-REFERENCES TO RELATED APPLICATIONS

This application is a continuation-in-part of copending U.S. patent application Ser. No. 09/517,906, filed Mar. 3, 2000, which is a continuation-in-part application of copending U.S. patent application Ser. No. 09/499,235, filed Feb. 7, 2000, which is a continuation-in-part of copending U.S. patent application Ser. No. 09/393,174, filed Sep. 9, 1999, and entitled “Neuronal Cell Death-Associated Molecules and Uses Therefor,” and is also based on U.S. Provisional Patent Application No. 60/161,188, filed Oct. 22, 1999, and entitled “Nucleic Acid Molecules Derived From Rat Brain and Programmed Cell Death Models,” each of which are hereby incorporated in their entirety by reference herein.

### FIELD OF THE INVENTION

The present invention relates to newly identified human and mouse programmed cell death (PCD) proteins having homology to a mammalian subtilase-like protein family, including prohormone convertases. The invention also relates to polynucleotides encoding the protein. The invention further relates to methods using the polypeptides and polynucleotides as a target for diagnosis and treatment in disorders mediated by or related to the protein. The invention further relates to drug-screening methods using the polypeptides and polynucleotides to identify agonists and antagonists for diagnosis and treatment. The invention further encompasses agonists and antagonists based on the polypeptides and polynucleotides. The invention further relates to procedures for producing the polypeptides and polynucleotides.

### BACKGROUND OF THE INVENTION

Programmed Cell Death

In multicellular organisms, homeostasis is maintained by balancing the rate of cell proliferation against the rate of cell death. Cell proliferation is influenced by numerous growth factors and the expression of proto-oncogenes, which typically encourage progression through the cell cycle. In contrast, numerous events, including the expression of tumor suppressor genes, can lead to an arrest of cellular proliferation.

In differentiated cells, a particular type of cell death called apoptosis occurs when an internal suicide program is activated. This program can be initiated by a variety of external signals as well as signals that are generated within the cell in response to, for example, genetic damage. Dying cells are eliminated by phagocytes, without an inflammatory response.

Programmed cell death is a highly regulated process (Wilson (1998) Biochem. Cell. Biol. 76:573-582). The death signal is then transduced through various signaling pathways that converge on caspase-mediated degradative cascades resulting in the activation of late effectors of morphological and physiological aspects of apoptosis, including DNA fragmentation and cytoplasmic condensation. In addition, regulation of programmed cell death may be integrated with regulation of energy, redox- and ion homeostasis in the mitochondria (reviewed by (Kroemer, 1998)), and/or cell-cycle control in the nucleus and cytoplasm (reviewed by (Choisy-Rossi and Yonish-Rouach, 1998; Dang, 1999; Kasten and Giordano, 1998)). Many mammalian genes regulating apoptosis have been identified as homologs of genes originally identified genetically in Caenorhabditis elegans or Drosophila melanogaster, or as human oncogenes. Other programmed cell death genes have been found by domain homology to known motifs, such as death domains, that mediate protein-protein interactions within the programmed cell death pathway.

The mechanisms that mediate apoptosis include, but are not limited to, the activation of endogenous proteases, loss of mitochondrial function, and structural changes, such as disruption of the cytoskeleton, cell shrinkage, membrane blebbing, and nuclear condensation due to degradation of DNA. The various signals that trigger apoptosis may bring about these events by converging on a common cell death pathway that is regulated by the expression of genes that are highly conserved.

Caspases (cysteine proteases having specificity for aspartate at the substrate cleavage site) are central to the apoptotic program. These proteases are responsible for degradation of cellular proteins that lead to the morphological changes seen in cells undergoing apoptosis. One of the human caspases was previously known as the interleukin-1β (IL-1β) converting enzyme (ICE), a cysteine protease responsible for the processing of pro-IL-1β to the active cytokine. Overexpression of ICE in Rat-1 fibroblasts induces apoptosis (Miura et al. (1993) Cell 75:653).

Many caspases and proteins that interact with caspases possess domains of about 60 amino acids called a caspase recruitment domain (CARD). Apoptotic proteins may bind to each other via their CARDs. Different subtypes of CARDs may confer binding specificity, regulating the activity of various caspases. (Hofmann et al. (1997) TIBS 22:155).

The functional significance of CARDs have been demonstrated in two recent publications. Duan et al. (1997) Nature 385:86 showed that deleting the CARD at the N-terminus of RAIDD, a newly identified protein involved in apoptosis, abolished the ability of RAIDD to bind to caspases. In addition, Li et al. (1997) Cell 91:479 showed that the N-terminal 97 amino acids of apoptotic protease activating factor-1 (Apaf-1) was sufficient to confer caspase-9-binding ability.

Thus, programmed cell death (apoptosis) is a normal physiological activity necessary to proper and differentiation in all vertebrates. Defects in apoptosis programs result in disorders including, but not limited to, neurodegenerative disorders, cancer, immunodeficiency, heart disease and autoimmune diseases (Thompson et al. (1995) Science 267:1456).

In vertebrate species, neuronal programmed cell death mechanisms have been associated with a variety of developmental roles, including the removal of neuronal precursors which fail to establish appropriate synaptic connections (Oppenheim et al. (1991) Annual Rev. Neuroscience 14:453-501), the quantitative matching of pre- and post-synaptic population sizes (Herrup et al. (1987) J. Neurosci. 7:829-836), and sculpting of neuronal circuits, both during development and in the adult (Bottjer et al. (1992) J. Neurobiol. 23:1172-1191).

Inappropriate apoptosis has been suggested to be involved in neuronal loss in various neurodegenerative diseases such as Alzheimer's disease (Loo et al. (1993) Proc. Natl. Acad. Sci. 90:7951-7955), Huntington's disease (Portera-Cailliau et al. (1995) J. Neurosc. 15:3775-3787), amyotrophic lateral sclerosis (Rabizadeh et al. (1995) Proc. Natl. Acad. Sci. 92:3024-3028), and spinal muscular atrophy (Roy et al. (1995) Cell 80:167-178).

In addition, improper expression of genes involved in apoptosis has been implicated in carcinogenesis. Thus, it has been shown that several “oncogenes” are in fact involved in apoptosis, such as in the Bcl family.

Accordingly, genes involved in apoptosis are important targets for therapeutic intervention. It is important, therefore, to identify novel genes involved in apoptosis or to discover whether known genes function in this process.

Subtilases/Proprotein Convertases

Subtilisin is an alkaline serine protease produced in various strains of Bacillus. Since it was first identified in B. subtilis, the enzyme was called “subtilisin.” Numerous variants have been identified and studied. This exoenzyme was found to belong to a large family of proteins spanning both prokaryotes and eukaryotes, variously designated, including “subtilases,” “subtilisin-related serine proteases,” and further designated “proprotein” or “prohormone convertases.”

Subtilisin is first produced as a precursor, pre- pro-subtilisin. The pre-sequence is a signal peptide which functions to export the protein across the membrane. The pro-sequence is essential for mediating proper folding of the mature catalytic region. Propeptide-mediated folding in subtilisin has been reviewed in Shinde et al. (In Subtilisin Enzymes; Practical Protein Engineering, 1996, Plenum Press, NY., pgs. 147-153). The function of the propeptide led to the concept of an intramolecular chaperone in subtilisin and subsequently in the subtilisin-related enzymes (see below).

The discovery of the fur locus led to the identification of the mammalian family of endoproteases, designated proprotein convertases (see above). These enzymes have a broad functional range and have been reviewed in Steiner, D. F. (1998) Current Opinion in Chemistry and Biology 2:31-39. Currently there are approximately seven members of the family. These catalyze the maturation of various peptide hormones and other precursor proteins and also are critical for virulence of pathogens, including bacterial and viral. Family members fall into two classes based on distribution, those expressed ubiquitously, such as furin and PACE4, and those with a more limited tissue distribution. Of the latter, prohormone convertase PC5/PC6 (differential nomenclature is a result of the naming by various groups) occurs mostly in gastrointestinal tissue. PC7/PC8/LPC is found in lymphoid tissue. PC1/sPC3 and PC2 are mainly restricted to tissues of neuroendocrine origin. PC4 is mainly localized to the testes.

The subtilases act within the secretory pathway to cleave polypeptide precursors at specific basic sites to generate their biologically active forms. Serum proteins, prohormones, receptors, zymogens, viral surface proteins, bacterial toxins, and others are activated by this route. Subtilisin-related serine proteases in the mammalian constitutive secretory pathway have been recently reviewed by Gensberg et al. (1998) Seminars in Cell and Dev. Biol. 9:11-17, summarized below.

These enzymes have also been referred to as Kex2-related serine proteases, or kexins because their discovery followed the characterization of Kex2, a calcium-dependent serine protease isolated from yeast. The subtilisin-related domain of Kex2 was found to be homologous to the human fur open reading frame. Moreover, the complete sequence of furin, the gene product of the fur gene, showed a more extensive similarity with Kex2. The preparation of degenerate PCR primers, designed to identify related sequences, led to the isolation of several other members of the family including PC2, PC1, PACE4, PC5/6, PC8/PC7/LPC and PC8. These family members share the same domain structure (shown in FIG. 1 of Gensberg et al.). All the members have a signal peptide that targets the protein to the secretory pathway. All members contain a pro-peptide that provides for correct folding of the active polypeptide and correct secretion from the endoplasmic reticulum. Cleavage of the propeptide is essential for activation and occurs autocatalytically in the endoplasmic reticulum, at least in the case of furin. All members contain a catalytic domain related to the bacterial subtilisins that contains, in the active site, ASP, HIS, and SER, and the oxyanion hole residue ASN, except in PC2 where ASP is substituted. All members also contain the P or middle domain, also referred to as the homo B domain. This domain plays a role in folding. Mutants in the P domain are not autocatalytically processed and remain in the endoplasmic reticulum. Most of the variation is found in the C-terminus. Furin, PC5/6B and PC7/8/LPC have C terminal transmembrane domains. The C terminal domains of furin, PACE4 and PC5/6 include a cysteine rich region. Further, all known family members contain potential glycosylation sites. Inhibition of glycosylation causes rapid degradation of PC1/3 and PC2 in the endoplasmic reticulum. Further, gene regulation and cellular and tissue distribution are unique for each family member.

Furin, later referred to as PACE, is a ubiquitous housekeeping proprotein processing endopeptidase of the constitutive secretory pathway. The furin transcript is expressed in all cell types and encodes a type I membrane protein predominantly localized to the trans-Golgi network and immature secretory granules of neuroendocrine and endocrine cells. The cytosolic tail of furin contains two signals that mediate localization in the late secretory pathway. These include an acid casein kinase II site (CPSDSEEDEG) that retains furin in the trans-Golgi network and a tyrosine motif (YKGI) that serves as a retrieval signal for furin that has escaped to the cell surface, cycling furin back to the trans-Golgi network via endosomes. A soluble form of furin, generated by cleavage N-terminal to the transmembrane domain, is shed from the cell.

The minimal recognition site for furin is R-XXR. However, the efficiency of cleavage may be modulated by the surrounding sequence. R-Q-P-R-G-W may be cleaved twice as efficiently as R-V-R-R-S-V, for example.

A wide range of protein precursors have been shown to be substrates for furin, such as parathyroid hormone-related peptide, pro-β-nerve growth factor, pro-albumin, complement pro-C3, semaphorins, pro-insulin-like growth factor 1A and integrin α-chain. Furin has been recently reviewed in detail in Nakayama (1997) Biochem. J. 327:625-635 and Molloy et al. (1999) Trends in Cell Biology 9:28-34, both of which are summarized below.

The expression of furin has been studied in rat development. mRNA is first detected in both endoderm and mesoderm in the primitive streak stage of embryogenesis. Subsequently a distinctly higher level of expression is observed in the heart and liver primordia. In mid and late gestational stages, furin is widely expressed in the peripheral tissues. The expression pattern of furin during embryogenesis is distinct from that of other ubiquitously expressed convertases and from neuroendocrine specific ones. This suggests that fturin plays a role in processing various proproteins, such as growth factor precursors, during development. Furin knock-out mice die by e11-12. The expression of furin is developmentally regulated and appears to control the growth and differentiation of cells such as pancreatic islet cells and gastric mucosal cells.

In furin, propeptide cleavage is not sufficient, although it is a prerequisite, for the activation of furin. After cleavage in the endoplasmic reticulum the propeptide remains associated with the mature furin moiety and functions as a potent autoinhibitor of the endoprotease. Upon transit through the endoplasmic reticulum, with a change of acidic conditions and calcium concentration, the propeptide is released, generating the active furin. This propeptide release requires a second cleavage at the ARG-GLY-VAL-THR-LYS-ARG site in the middle of the propeptide. Mutations in this sequence result in an endoprotease that cannot be activated by acid or calcium treatment in vitro.

Various substrates of furin and sequences around the cleavage sites of precursor proteins are shown in Nakayama, above. Furin is proposed to be responsible for processing precursors of constitutively secreted proteins rather than peptide hormones and neuropeptides. These include growth factors, their receptors, plasma proteins involved in blood clotting and complement systems, matrix metalloproteases, viral envelope glycoproteins, and bacterial exotoxins. Furin preferentially recognizes the cleavage sequence ARG-XAA-(LYS/ARG)-ARG. However, cleavage sites of some precursors cleaved by furin do not fully fit this consensus sequence. Accordingly, Nakayama has proposed the following sequence rules governing cleavage by furin: (1) An ARG residue is essential at the P₁ position; (2) In addition to the P₁ ARG, at least two out of the three residues at P₂, P₄ and P₆ are required to be basic for efficient cleavage; (3) At P₁ position an amino acid with a hydrophobic aliphatic side chain is not suitable. The cleavage site specificity determined by coexpression studies is in agreement with that determined by in vitro studies using purified recombinant soluble forms of furin.

Since furin cleavage is essential to produce a wide variety of biologically active proteins, it has been proposed that mutation of the cleavage site may result in genetic disorders. It has been reported that a severe form of hemophilia B is correlated with mutation of the P₄ ARG residue to GLN in pro-factor IX. There have also been many reports of hemophilia B cases with mutations of the P₄, P₂ or P₁ basic residue of pro-factor IX. Further, subjects with extreme insulin resistance were reported to have a mutation of the P₁ ARG residue to SER at the cleavage site of insulin proreceptor.

As discussed above, furin function is implicated in productive viral infection. Proteolytic activation of envelope glycoproteins is necessary for the entry of viruses into host cells. In some cases it has been shown that the cleavability of the envelope glycoproteins is an important determinant for viral pathogenecity. For example, proteins required for infectivity of mammalian influenza viruses and avirulent avian-influenza viruses, which can cause local infection, are susceptible to proteolytic cleavages only in specific cell types, such as in the respiratory and alimentary tract. In contrast, virulent avian-influenza viruses that cause systemic infection are cleaved in a variety of host cells. Similarly, avirulent and virulent Newcastle disease viruses cause local and systemic infections, respectively. A relationship has been suggested between viral pathogenecity and the cleavage site sequence of envelope glycoprotein precursors. In vitro experiments using purified furin have shown that furin is involved in cleavage of the glycoprotein precursors of virulent viruses. Accordingly, the widespread expression of furin can account for systemic infections by virulent viruses. Furin has also been implicated in the activation of HIV-1 gp160. Furin is also expressed in CD4⁺ cell lines. However, other proteases may also be involved in gp160 cleavage.

The range of proproteins activated by furin (Table 1 in Molloy et al., above, incorporated herein by reference) is extensive and indicates an importance in fundamental biological processes. Furin is involved in cellular signaling at both the juxtacrine (for example, cell adhesion factors) and paracrine (for example, growth factors and receptors) levels. It can regulate the composition of the extracellular matrix (for example, processing of matrix components and activation of matrix metalloproteases) and contributes to the processes of embryonic induction.

As indicated above, proprotein convertases typically cleave their substrates on the C-terminal side of paired basic amino acids (for example, LYS-ARG^{↓}, -ARG-ARG^{↓}. Furin generally requires an additional ARG at the P₄ position for efficient cleavage of substrates (-ARG-X-LYS/ARG-ARG^{↓}-). The residues that are C-terminal to the cleavage site (P′) also affect processing efficiency and possibly specificity.

As indicated, the propeptide is a multifunctional domain directing the compartment-specific activation of furin. Because of the role in directing the correct folding of the mature peptide, the propeptide functions as an intramolecular or steric chaperone. Autoproteolytic cleavage occurs at ↓R-T-K-R₁₀₇↓R-G-V-T-K-R₇₅. In addition, the prosequence contains an autoinhibitory domain. The catalytic domain is known to contain high and middle affinity calcium binding sites. The P domain is necessary for the activity of furin and other proprotein convertases. The P domain also functions in pH and calcium modulation. This domain also contains a conserved RGD integrin binding motif. Mutation of this site in PC1/3 disrupts proenzyme maturation and catalytic activity. In furin, this motif may function in matrix association.

The transmembrane domain (in furin) is followed by a cytosolic domain that contains sorting information, including multiple clathrin-coat-recruitment motifs that control internalization, budding from the trans-Golgi network, and polarized sorting. This region also contains a cluster of acidic amino acids that directs phosphorylation-state-specific trans-Golgi network localization and endosomal sorting, and a membrane proximal region that tethers fturin to the cortical cytoskeleton.

The trafficking of fturin between the trans-Golgi network, cell surface, and endosomes is directed by defined sequence motifs in the cytosolic domain. Localization to the trans-Golgi network and the endosomal routing of furin is dependent upon the phosphorylation state of the acidic cluster. Dephosphorylated furin is delivered to the trans-Golgi network, whereas the phosphorylated enzyme is recycled to the plasma membrane. Furin is retained in a bi-cycling loop by casein kinase II-mediated phosphorylation of serine residues in the acidic cluster motif. Dephosphorylation regulates movement between the loops. Furin molecules delivered to the cell surface can be tethered via binding to a component of the cortical actin cytoskeleton. Localization of furin to the trans-Golgi network requires the cooperative effect of the phosphorylated acidic cluster motif together with one or more clathrin-coated pit recruitment signals. This bi-cycling has been described in Molloy et al, above.

The furin activation pathway is essentially as follows: (1) Initial synthesis as a zymogen within the neutral pH environment of the endoplasmic reticulum; (2) Rapid autoproteolytic cleavage of the propeptide at the consensus furin site ARG-THR-LYS-ARG₁₀₇; (3) Endoplasmic reticulum to Golgi transport, along with propeptide cleavage, where the cleaved propeptide remains associated with the enzyme and functions as a potent autoinhibitor; (4) Within the mildly acidic environment of the trans-Golgi network/endosomal system, the propeptide is cleaved autoproteolytically at a P1/P2/P6 ARG-containing furin site (ARG-GLY-VAL-THR-LYS-ARG^{75↓}), releasing the propeptide fragments and thus providing active furin; (5) Within the late secretory pathway, independent of the activation state, furin is cleaved upstream of its transmembrane domain, potentially functioning in the processing of extracellular substrates (for example, extracellular matrix components).

As indicated, furin is implicated in early development. Disruption of the mouse gene encoding furin results in embryonic lethality. This is associated with several defects, including failure of the heart tube to fuse or to undergo looping morphogenesis and failure of the embryos to undergo axial rotation. The results are consistent with a role for furin in maturation of members of the TGFβ family, particularly bone morphogenic proteins and nodal-related proteins.

There is also a relationship between furin processing and regulation of the extracellular matrix. Specifically, the soluble (shed) furin is implicated in processing of extracellular matrix proteins, for example fibrillin and zona pellucida proteins. It also has a role in the activation of matrix metalloproteases, such as BMP-1/procollagen, C-protease and stromelysin-3. Changes in furin-dependent matrix metalloprotease activation can contribute to the metastatic capacity of tumors. The observation that stromelysin-3 activity corresponds with tumor invasiveness supports this possibility.

Finally, as indicated, furin has been associated with pathogenic virulence. Numerous pathogens require cleavage by furin of viral envelope glycoproteins and bacterial toxins for their virulence. Processing is apparently a key determinant in viral tropism. For example, the cleavage site in Ebola virus glycoprotein GP is coupled to the lethality of the virus in humans. Also, a fatal respiratory illness has been traced to a specific strain of avian influenza A. Sequence analysis of genes encoding the HA gene showed a consistent alteration in the viral genomes, the generation of a second consensus furin site.

PC1 and PC2, although not as well characterized as furin, have also been analyzed in relative detail. PC1 and PC2 are primarily expressed in endocrine and neural cells, mostly localizing within the trans-Golgi network or dense core secretory granules. These molecules have been reviewed recently by Muller et al. (2000) Progress in Nucleic Acid Research and Molecular Biology 63:60-109. These enzymes participate in the regulated proteolysis of prohormones and are designated prohormone convertases (PC). A human patient exhibiting obesity, hyperproinsulinemia, and hypocortisolemia phenotypes was shown to have nucleotide mutations in each allele of the PC1 gene, resulting in an inactive convertase. In addition, homozygous PC2 null mice are viable but exhibit a hypoglycemia phenotype. PC1 substrates include but are not limited to POMC (β-LPH, ACTH), proinsulin, proTRH, proENK, proDyn, proglucagon, prorenin, proMCH, proNT, and proCCK. PC2 substrates include but are not limited to POMC, proinsulin, proglucagon, proNT, proENK, proLHRH, proDyn, and proCCK.

Cellular expression and subcellular localization of prohormone convertases have also been reviewed in Seidah, et al. (1997) Current Opinion in Biotechnology 8:602-607, summarized herein.

PC4 is exclusively expressed in germ cells of the testes. Homozygous PC4 null mice are viable but have reduced male fertility.

PC5 and PACE4 are widely expressed and detected during early embryonic development. In the adult, PC5 is highly expressed in gut, endothelial and Sertoli cells and in the adrenal cortex.

Accordingly, subtilases/proprotein convertases (“subtilases”) are a major target for drug action and development. Thus, it is valuable to the field of pharmaceutical development to identify and characterize previously unknown subtilases or subtilase-like proteins. The present invention advances the state of the art by providing previously unidentified human, mouse, and rat subtilase-like proteins that are regulated in programmed cell death.

### SUMMARY OF THE INVENTION

It is an object of the invention to identify novel subtilases.

It is a further object of the invention to provide novel subtilase polypeptides that are useful as reagents or targets in subtilase assays applicable to treatment and diagnosis of subtilase-mediated or -related disorders.

It is a further object of the invention to provide polynucleotides corresponding to the novel subtilase polypeptides that are useful as targets and reagents in subtilase assays applicable to treatment and diagnosis of subtilase-mediated or -related disorders and useful for producing novel subtilase polypeptides by recombinant methods.

A specific object of the invention is to identify compounds that act as agonists and antagonists and modulate the expression of the novel protein.

A further specific object of the invention is to provide compounds that modulate expression of the protein for treatment and diagnosis of disorders related to the subtilase-like protein.

The invention is thus based on the identification of novel human, mouse, and rat subtilase-like proteins. The amino acid sequence is shown in SEQ ID NOS:1, 3, 5, and 7. The nucleotide sequence is shown in SEQ ID NOS:2, 4, 6, and 8.

The invention provides isolated subtilase-like polypeptides, including a polypeptide having an amino acid sequence shown in SEQ ID NOS:1, 3, 5, or 7.

The invention also provides variant polypeptides having an amino acid sequence that is substantially homologous to an amino acid sequence shown in SEQ ID NOS:1, 3, 5, or 7.

The invention also provides variant nucleic acid sequences that are substantially homologous to a nucleotide sequence shown in SEQ ID NOS:2, 4, 6, or 8.

The invention also provides fragments of a polypeptide shown in SEQ ID NOS:1, 3, 5, or 7 and nucleotide sequence shown in SEQ ID NOS:2, 4, 6, or 8, as well as substantially homologous fragments of the polypeptide or nucleic acid.

The invention also provides mutants and truncations of the molecules of the invention. Non-limiting examples of such mutants and truncations are set forth in SEQ ID NOS:9, 10, 11, 12, 13, 14, 15, 16, 17, 18, 19, 20, 21, 22, 23, 24, 25, and 26.

The invention further provides nucleic acid constructs comprising the nucleic acid molecules described herein. In a preferred embodiment, the nucleic acid molecules of the invention are operatively linked to a regulatory sequence.

The invention also provides vectors and host cells for expressing the subtilase-like nucleic acid molecules and polypeptides, and particularly recombinant vectors and host cells.

The invention also provides methods of making the vectors and host cells and methods for using them to produce the subtilase-like nucleic acid molecules and polypeptides.

The invention also provides antibodies or antigen-binding fragments thereof that selectively bind the subtilase-like polypeptides and fragments.

The invention also provides methods of screening for compounds that modulate expression or activity of the subtilase-like polypeptides or nucleic acid (RNA or DNA).

The invention also provides a process for modulating the subtilase-like polypeptide or nucleic acid expression or activity, especially using the screened compounds. Modulation may be used to treat conditions related to aberrant activity or expression of the subtilase-like polypeptides or nucleic acids.

The invention also provides assays for determining the activity of or the presence or absence of the polypeptides or nucleic acid molecules in a biological sample, including for disease diagnosis.

The invention also provides assays for determining the presence of a mutation in the polypeptides or nucleic acid molecules, including for disease diagnosis.

In still a further embodiment, the invention provides a computer readable means containing the nucleotide and/or amino acid sequences of the nucleic acids and polypeptides of the invention, respectively.

### DESCRIPTION OF THE DRAWINGS

FIGS. 1(A-C) shows the human nucleotide sequence NARC1A (SEQ ID NO:2) and the deduced amino acid sequence (SEQ ID NO:1).

FIG. 2 shows an analysis of the human NARC1A amino acid sequence: αβturn and coil regions; hydrophilicity; amphipathic regions; flexible regions; antigenic index; and surface probability plot.

FIG. 3 shows a hydrophobicity plot of the human NARC1A protein.

FIGS. 4(A-B) shows an analysis of the human NARC1A protein open reading frame for amino acids corresponding to specific functional sites. For the phosphorylation and myristoylation sites, the actual modified residue is the first amino acid.

FIGS. 5(A-E) shows the rat NARC1 nucleotide sequence (SEQ ID NO:4) and the deduced amino acid sequence (SEQ ID NO:3). Note that the numbers on the left refer to the number of amino acids or nucleotides in the preceding line.

FIGS. 6(A-D) shows the mouse NARC1 nucleotide sequence (SEQ ID NO:6) and the deduced amino acid sequence (SEQ ID NO:5) designated mouse NARC1. This gene is a murine ortholog of the rat and human NARC1 sequences above. Note that the numbers on the left refer to the number of amino acids or nucleotides in the preceding line.

FIGS. 7(A-E) shows the human NARC1C nucleotide sequence (SEQ ID NO:8) and the deduced amino acid sequence (SEQ ID NO:7). Note that the numbers on the left refer to the number of amino acids or nucleotides in the preceding line.

FIG. 8 shows the result of experiments designed to characterize transcriptional characteristics for the rat NARC1 gene. The top panel summarizes results from transcription profiling experiments performed on Smart Chip I for the rat NARC1. Hybridization signals (gene expression intensities) are plotted on the Y-axis. Experiments are listed along the X-axis in the following order from left to right: 1, 3, 6, 12, 24 h post serum add-back (SA), 1, 3, 6, 12, 24 h post KCl plus serum withdrawal (KCl/S), 1, 3, 6, 12 h post sham KCl treatment (KCl-C), 1, 3, 6, 12 h KCl withdrawal (KCl), 2, 4, 8, 12 h post sham kainic acid treatment (KT C), and 2, 4, 8, 12, h post kainic acid treatment (KT). The RT-PCR panel shows confirmation of the transcription profiling result at 3 h post KCl plus serum withdrawal (>4× upregulation) (See Materials and Methods section of U.S. Provisional Patent Application No. 60/161,188). Rat NARC1 was originally cloned by differential display (RADE) (U.S. patent application Ser. No. 09/393,174). The transcript size of rat NARC1 measured by a multiple tissue Northern (bottom panel) was 3.4 kb. The result of multiple tissue Northern indicated high levels of rat NARC1 expression in the liver, and less expression in the kidney and testes. The signal in testes indicates the presence of a shorter isoform consistent with the size of human NARC1A (FIG. 1). Human NARC1C (FIG. 7) is an ortholog of the larger rat splice variant.

### DETAILED DESCRIPTION OF THE INVENTION

Polypeptides

The invention is based on the identification of a novel human subtilase-like protein which is regulated in programmed cell death (apoptosis).

Programmed cell death (PCD) in rat cerebellar granule neurons (CGNs) induced by potassium (K⁺) withdrawal has been shown to depend on de novo RNA synthesis. This transcriptional component of CGN programmed cell death was characterized using a custom-built brain-biased cDNA array representing over 7000 different rat genes. Consistent with carefully orchestrated mRNA regulation, the profiles of 234 differentially expressed genes segregated into distinct temporal groups (immediate early, early, middle, and late) encompassing genes involved in distinct physiological responses including cell-cell signaling, nuclear reorganization, apoptosis, and differentiation. A set of 64 genes, including 22 novel genes, were regulated by both K⁺ withdrawal and kainate treatment. Thus, by using array technology, physiological responses at the transcriptional level were characterized and novel genes induced by multiple models of programmed cell death were identified. The rat NARC1 was among these genes.

The above experimental subject matter was disclosed in U.S. Provisional Patent Application No. 60/161,188, filed Oct. 22, 1999, entitled “Nucleic Acid Molecules Derived From Rat Brain and Programmed Cell Death Models.” The novel genes included the gene designated “NARC1.” This gene is further disclosed in U.S. Provisional Patent Application No. 60/099,616, entitled “Neuronal Cell Death Associated Molecules and Uses Therefor”, now U.S. patent application Ser. No. 09/393,174. In the present application, a human NARC1 ortholog has been identified, designated NARC1A.

Accordingly, the human ortholog was cloned from a cDNA library of human keratinocytes treated with KGF, GF and cycloheximide.

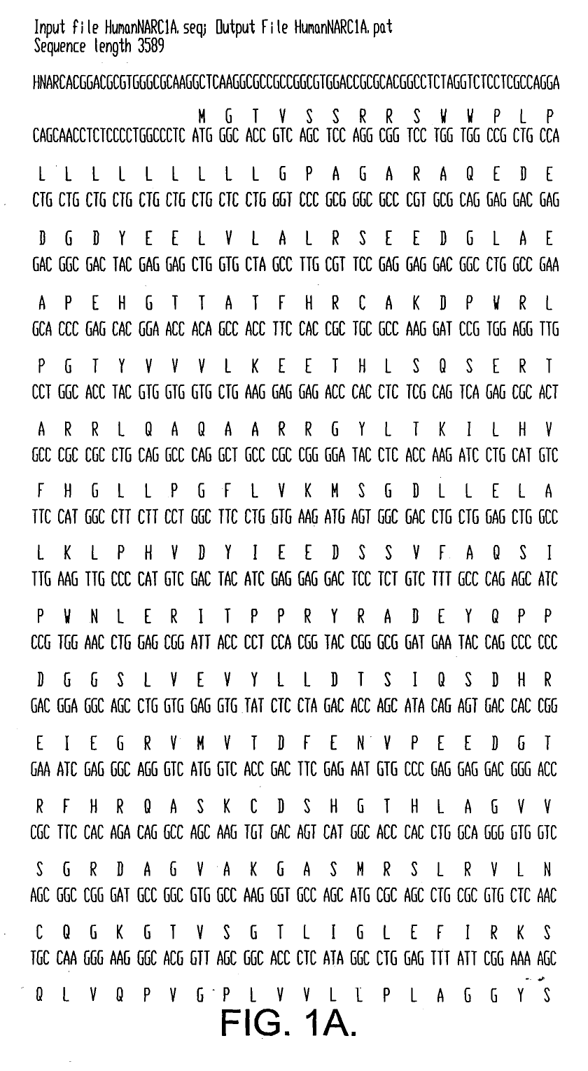

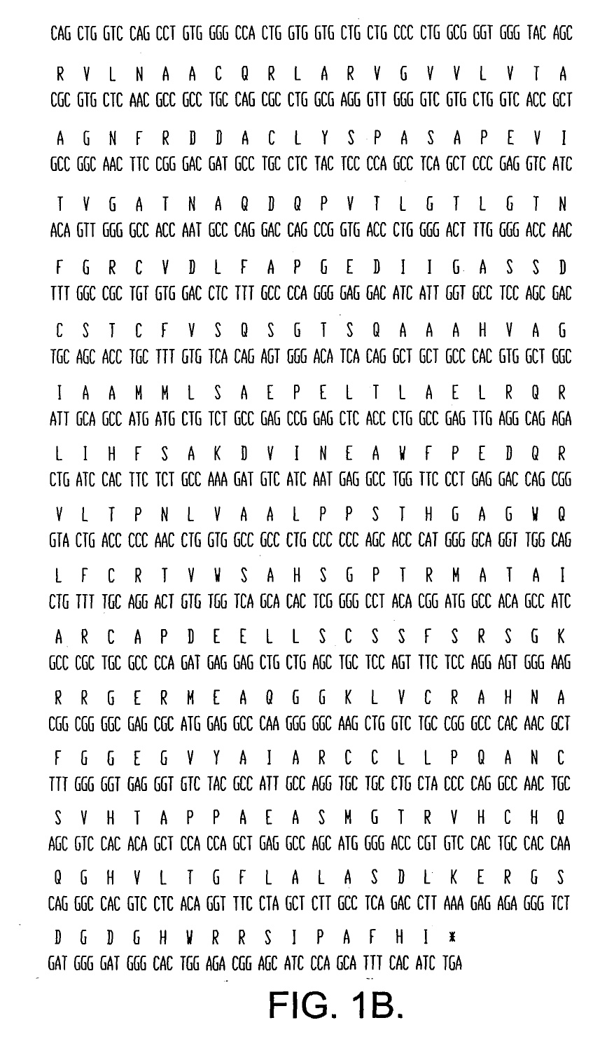

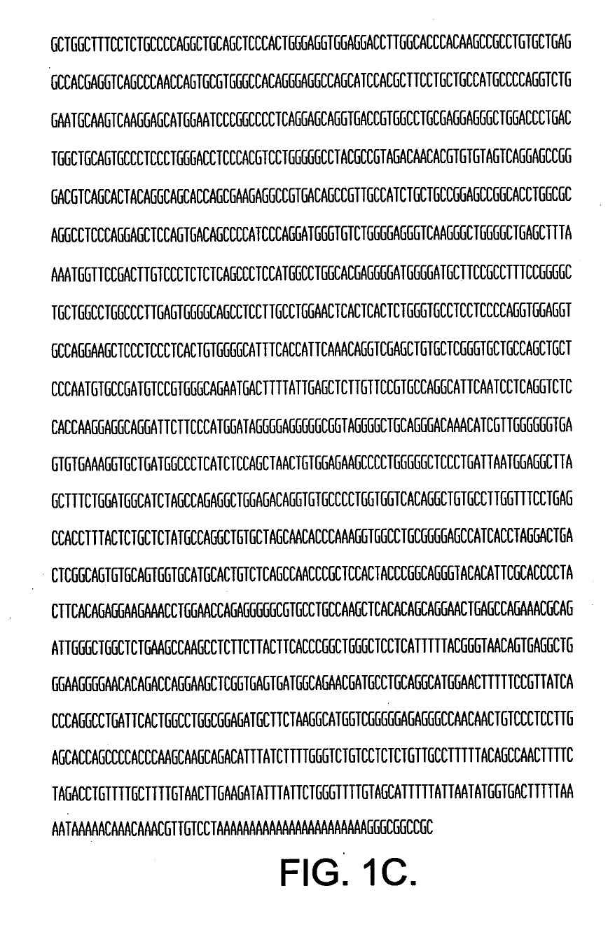

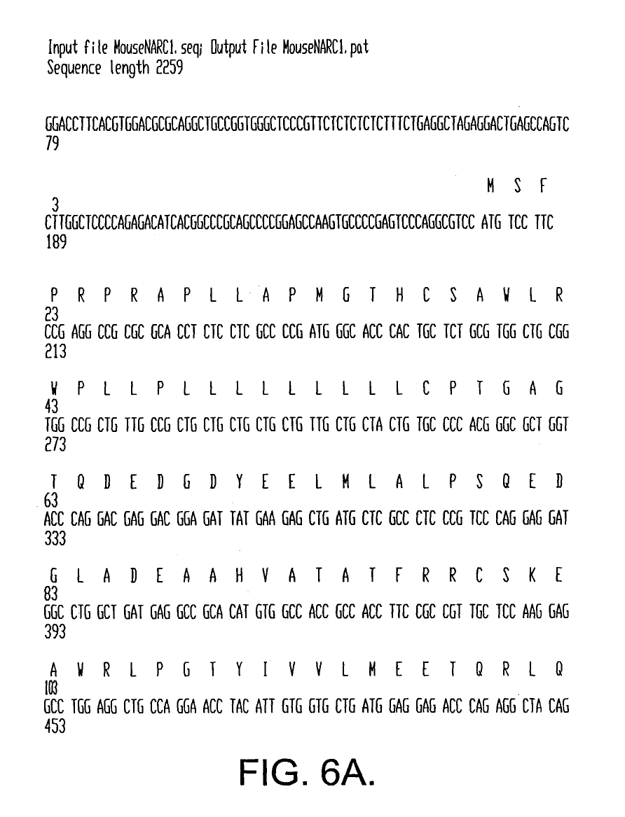

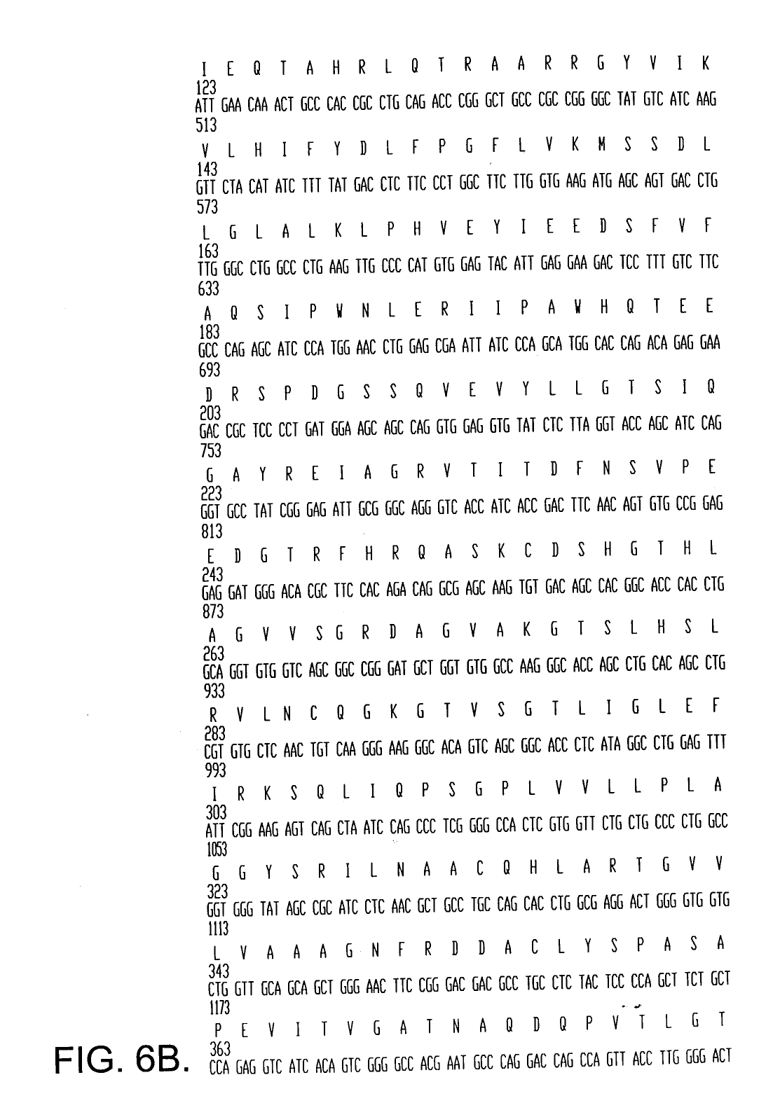

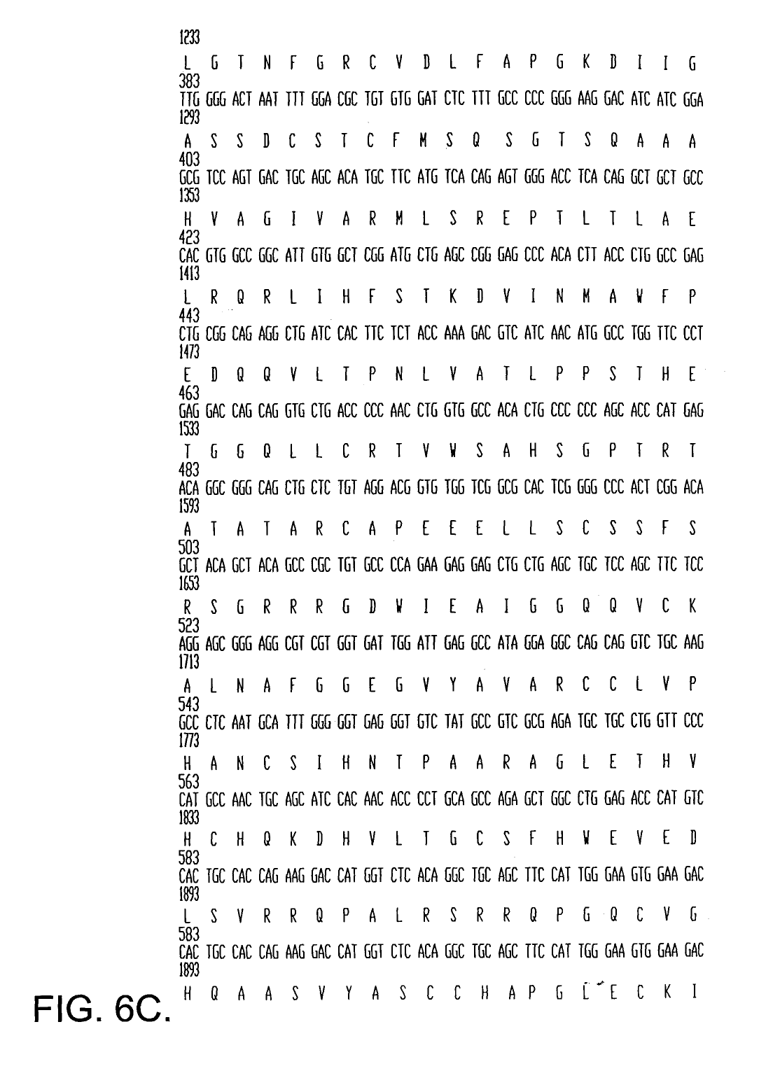

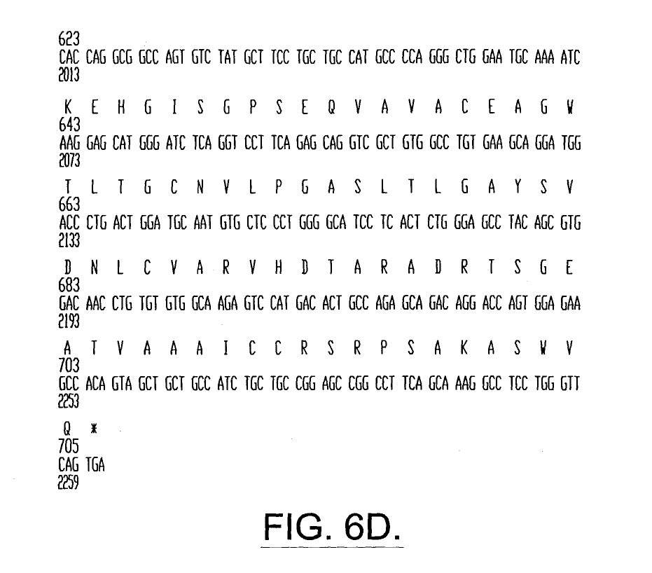

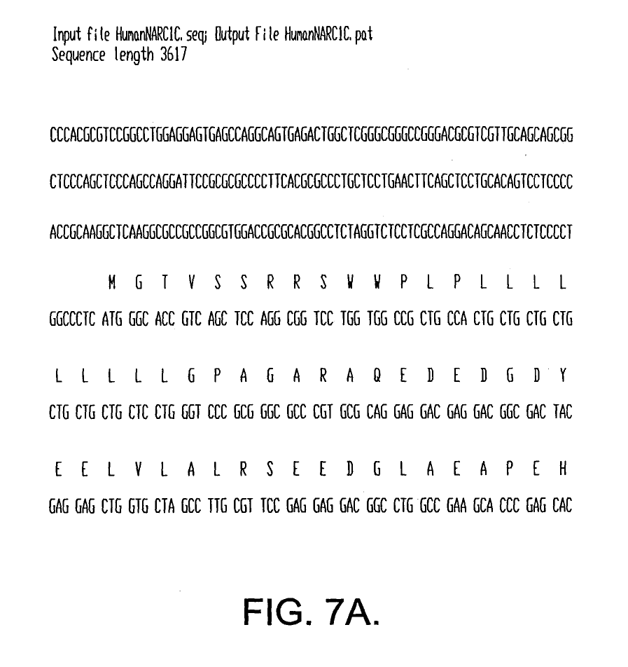

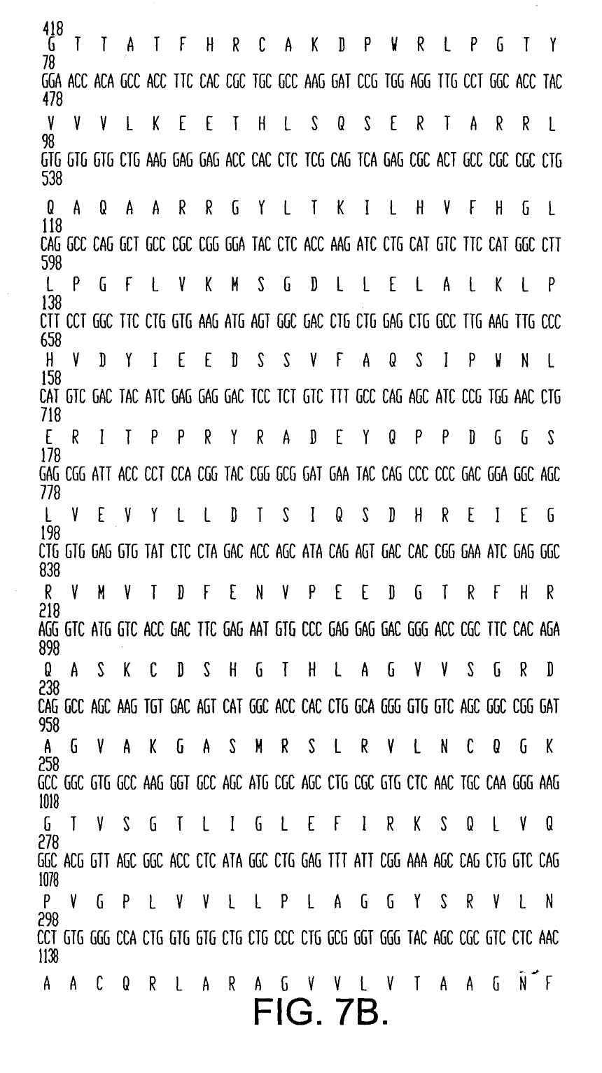

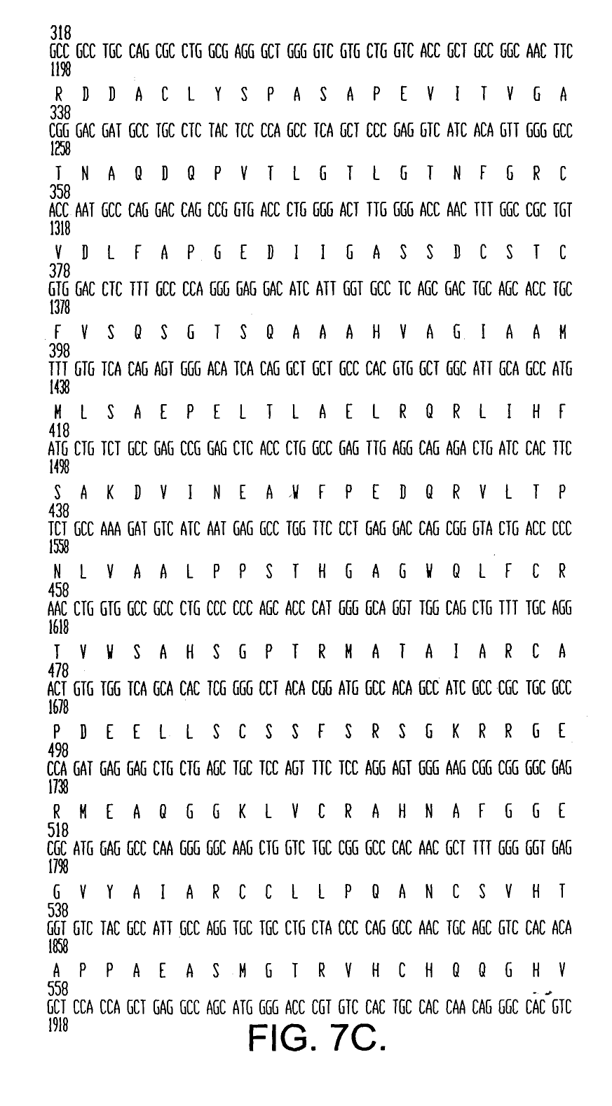

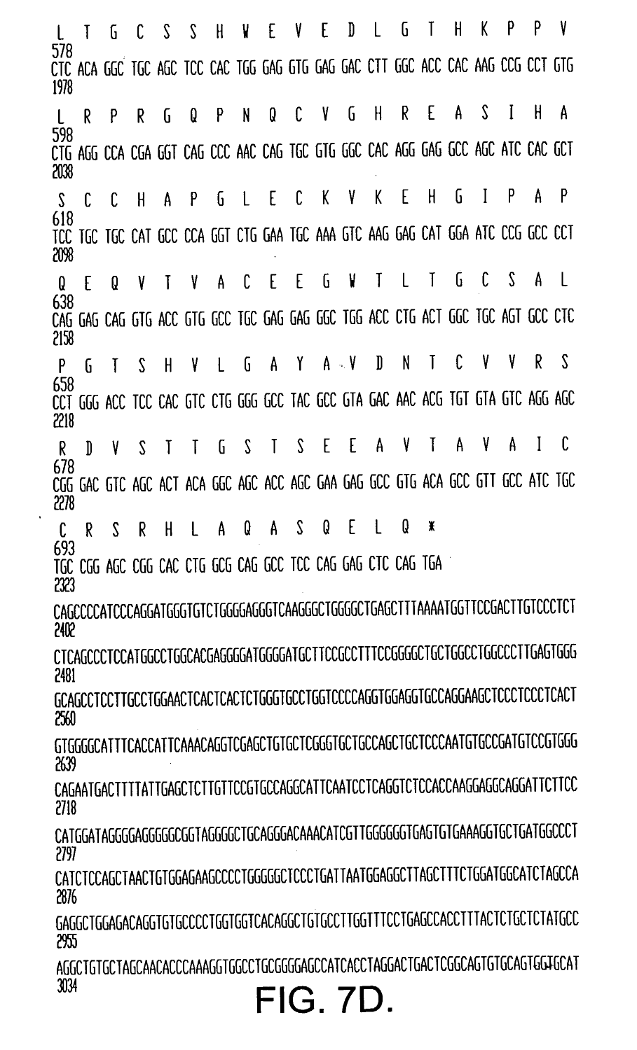

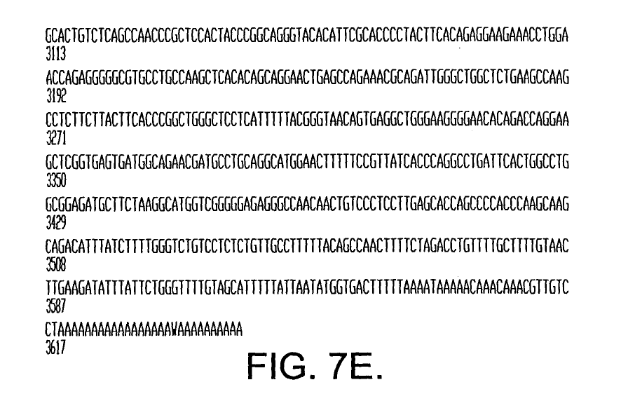

The invention thus relates to novel human, mouse, and rat subtilase-like proteins having a deduced amino acid sequence shown in FIGS. 1, and 5-7 (SEQ ID NOS:1, 3, 5, and 7).

“Subtilase-like polypeptide” or “subtilase-like protein” refers to a polypeptide in SEQ ID NOS:1, 3, 5, or 7. The term “subtilase-like protein” or “subtilase-like polypeptide,” however, further includes the numerous variants described herein, as well as fragments derived from the full-length subtilase-like proteins and their variants.

The present invention thus provides an isolated or purified subtilase-like polypeptide and variants and fragments thereof.

Based on a BLAST search, highest homology of the human protein in FIG. 1 was shown to an aqualysin precursor. Homology was also shown to subtilase-like proteins in other organisms and to prohormone convertases.

The human protein in FIG. 1 is expressed in tissues that include but are not limited to testes and liver. High relative expression occurs in liver.

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.”

The subtilase-like polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of the subtilase-like protein having less than about 30% (by dry weight) other proteins (i.e., contaminating protein), less than about 20% other proteins, less than about 10% other proteins, or less than about 5% other proteins. When the polypeptide is recombinantly produced, it can also be substantially free of culture medium, i.e., culture medium represents less than about 20%, less than about 10%, or less than about 5% of the volume of the protein preparation.

A subtilase-like polypeptide is also considered to be isolated when it is part of a membrane preparation or is purified and then reconstituted with membrane vesicles or liposomes.

The language “substantially free of chemical precursors or other chemicals” includes preparations of the subtilase-like polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the subtilase-like polypeptide comprises an amino acid sequence shown in SEQ ID NOS:1, 3, 5, or 7. However, the invention also encompasses sequence variants. Variants include a substantially homologous protein encoded by the same genetic locus in an organism, i.e., an allelic variant. The human subtilase-like protein in FIG. 1 has been mapped to human chromosome 1p32. Two diseases are known to map at this locus. These include muscle-eye-brain disease at 1p34-p32 (MEB) and Bartter Syndrome, infantile, with sensorineural deafness (BSND), at 1p31, both of which are discussed in more detail herein below.

Variants also encompass proteins derived from other genetic loci in an organism, but having substantial homology to a subtilase-like protein of SEQ ID NOS:1, 3, 5, or 7. Variants also include proteins substantially homologous to the subtilase-like protein but derived from another organism, i.e., an ortholog. Variants also include proteins that are substantially homologous to the subtilase-like protein that are produced by chemical synthesis. Variants also include proteins that are substantially homologous to the subtilase-like protein that are produced by recombinant methods. It is understood, however, that variants exclude any amino acid sequence disclosed prior to the invention.

As used herein, two proteins (or a region of the proteins) are substantially homologous when the amino acid sequences are at least about 50-55%, 55-60%, 60-65%, 65-70%, 70-75%, typically at least about 80-85%, and most typically at least about 90-95% or more homologous. A substantially homologous amino acid sequence, according to the present invention, will be encoded by a nucleic acid sequence hybridizing to the nucleic acid sequence, or portion thereof, of a sequence shown in SEQ ID NOS:2, 4, 6, or 8 under stringent conditions as more fully described below.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The invention also encompasses polypeptides having a lower degree of identity but having sufficient similarity so as to perform one or more of the same functions performed by the subtilase-like protein. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al. (1990) Science 247:1306-1310.

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLAST) can be used. See www.ncbi.nlm.nih.gov. In one embodiment, parameters for sequence comparison can be set at score=100, wordlength=12, or can be varied (e.g., W=5 or W=20).

In a preferred embodiment, the percent identity between two amino acid sequences is determined using the Needleman et al. (1970) J. Mol. Biol. 48:444-453 algorithm which has been incorporated into the GAP program in the GCG software package (available at www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is' determined using the GAP program in the GCG software package (Devereux et al. (1984) Nucleic Acids Res. 12(1):387) (available at www.gcg.com), using a NWSgapdna.CMP matrix and a gap weight of 40, 50, 60, 70, or 80 and a length weight of 1, 2, 3, 4, 5, or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al. (1994) Comput. Appl. Biosci. 10:3-5; and FASTA described in Pearson et al. (1988) PNAS 85:2444-8.

A variant polypeptide can differ in amino acid sequence by one or more substitutions, deletions, insertions, inversions, fusions, and truncations or a combination of any of these.

Variant polypeptides can be fully functional or can lack function in one or more activities. Thus, in the present case, variations can affect the function, for example, of one or more of the regions corresponding to the prodomain, catalytic domain, P domain, cysteine-rich domain, transmembrane domain, and cytosolic domain. Functions that can be affected include but are not limited to autoproteolysis, intracellular chaperone function, propeptide processing, and autoinhibitory function in the prodomain, the ability to be modulated by pH and calcium, cell adhesion/integrin-binding, and collateral catalytic activity in the P domain, and cell surface tethering, TGN localization, and casein kinase II phosphorylation in the cytosolic domain.

Fully functional variants typically contain only conservative variation or variation in non-critical residues or in non-critical regions. Functional variants can also contain substitution of similar amino acids, which results in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for the subtilase-like polypeptide. This includes preventing immunogenicity from pharmaceutical formulations by preventing protein aggregation.

Useful variations further include alteration of catalytic activity. For example, one embodiment involves a variation in the catalytic domain that results in more or less affinity for the substrate propeptide. Another variation in this domain would result in greater or lesser rates of hydrolysis of propeptide substrate. A further variation in the catalytic domain results in altered specificity for the substrate propeptide, for example affinity for another (different) substrate which can include affinity for additional substrates or loss of specificity for the native substrate. Another variation is alteration of autocatalytic activity. This in turn would affect intramolecular chaperone functions. A further variation is one that affects the ability to be activated, for example by pH or calcium. A further variation includes a variation in the targeting potential. For example, a variation in the ability to be phosphorylated by casein kinase II could affect intracellular trafficking. Another variation involves an alteration in the acidic cluster motif in the cytosolic domain which results in changes in intracellular localization. A further variation includes one that prevents truncation of the molecule and hence affects extracellular matrix-associated functions. Another useful variation provides a fusion protein in which one or more domains or subregions are operationally fused to one or more domains or subregions from a different subtilase, subtilase-like protein, prohormone convertase, or proprotein convertase.

Amino acids that are essential for function can be identified by methods known in the art, such as site-directed mutagenesis or alanine-scanning mutagenesis (Cunningham et al. (1985) Science 244:1081-1085). The latter procedure introduces single alanine mutations at every residue in the molecule. The resulting mutant molecules are then tested for biological activity, such as autoproteolysis or propeptide proteolysis in vitro, or in vitro or in vivo activity that is dependent upon autoproteolytic cleavage or propeptide proteolytic cleavage, such as cell proliferation, development, V-ATPase function, extracellular matrix formation, inflammation, apoptosis/programmed cell death, and viral and bacterial pathogenesis and toxicity, as well as other effects disclosed herein. Sites that are critical, for example, for propeptide binding, can also be determined by structural analysis such as crystallization, nuclear magnetic resonance or photoaffinity labeling (Smith et al. (1992) J. Mol. Biol. 224:899-904; de Vos et al. (1992) Science 255:306-312).

Substantial homology can be to the entire nucleic acid or amino acid sequence or to fragments of these sequences.

In one embodiment, a polypeptide of the present invention has at least 60%, 70%, 80%, 90%, 91%, 92%, 93%, 94%, 95%, 96%, 97%, 98%, 99% or greater identity to a polypeptide sequence shown in SEQ ID NOS:1, 3, 5, 7, 9, 11, 13, 15, 17, 19, 21, 23, or 25.

The invention also includes polypeptide fragments of the subtilase-like protein. Fragments can be derived from an amino acid sequence shown in SEQ ID NOS:1, 3, 5, or 7. However, the invention also encompasses fragments of the variants of the subtilase-like proteins as described herein.

The fragments to which the invention pertains, however, are not to be construed as encompassing fragments that may be disclosed prior to the present invention.

Accordingly, a fragment can comprise at least about 10, 15, 20, 25, 30, 35, 40, 45, 50 or more contiguous amino acids. Fragments can retain one or more of the biological activities of the protein, for example the ability to bind to or hydrolyze substrate, as well as fragments that can be used as an immunogen to generate antibodies.

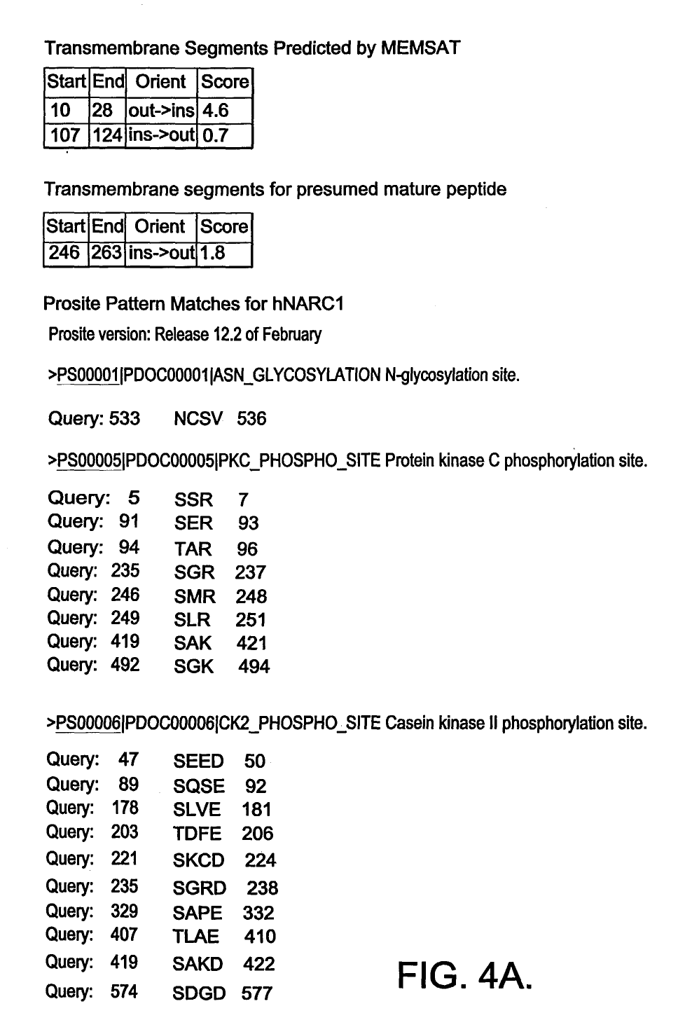

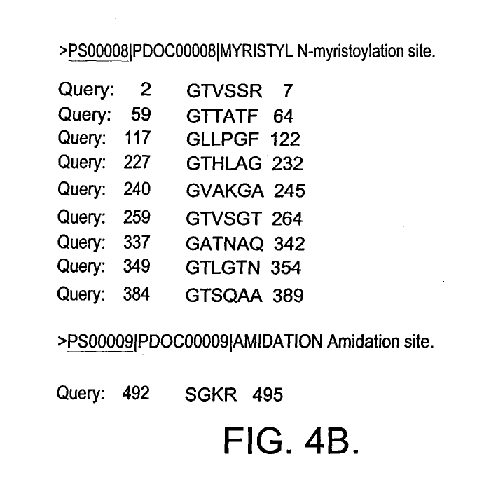

Biologically active fragments (peptides which are, for example, 5, 7, 10, 12, 15, 20, 30, 35, 36, 37, 38, 39, 40, 50, 100 or more amino acids in length) can comprise a domain or motif, e.g., as discussed above, as well as functional sites shown in FIG. 4 herein.

Such domains or motifs can be identified by means of routine computerized homology searching procedures or by routine assays, such as those disclosed herein.

Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived.

These regions can be identified by well-known methods involving computerized homology analysis.

In one embodiment, a fragment contains 56-746 of SEQ ID NO:3, set forth herein as SEQ ID NO:9.

Fragments include proteins with substitutions at particular amino acid residues. Techniques for the generation of site directed mutants are known in the art and set forth, for example, in Ausubel et al. (1998) Current Protocols in Molecular Biology, John Wiley & Sons.

In one embodiment, the histidine residue at position 225 of SEQ ID NO:9 is replaced by another amino acid residue. In another embodiment, the histidine residue at position 225 of SEQ ID NO:9 is replaced by tryptophan (SEQ ID NO:11). The activity of this molecule is described below, in the Examples.

In another embodiment, the serine residue at position 385 of SEQ ID NO:9 is replaced by another amino acid residue. In another embodiment, the serine residue at position 385 of SEQ ID NO:9 is replaced by alanine (SEQ ID NO:13). The activity of this molecule is described below, in the Examples.

In another embodiment, both the histidine residue at position 225 of SEQ ID NO:9 and the serine residue at position 385 of SEQ ID NO:9 are replaced by different amino acid residues. In another embodiment, both the histidine residue at position 225 of SEQ ID NO:9 and the serine residue at position 385 of SEQ ID NO:9 is replaced by tryptophan and alanine, respectively (SEQ ID NO:15). The activity of this molecule is described below, in the Examples.

Fragments can include truncations and deletion mutants. Techniques for the generation of such molecules are known in the art and set forth, for example, in Ausubel et al. (above).

In one embodiment, the polypeptide set forth in SEQ ID NO:9 is truncated immediately after the methionine residue at position 425 of SEQ ID NO:9, set forth herein as SEQ ID NO:17. The activity of this molecule is described below, in the Examples. In another embodiment, the polypeptide set forth in SEQ ID NO:9 is truncated immediately after the glutamine residue at position 453 of SEQ ID NO:9, set forth herein as SEQ ID NO:19. The activity of this molecule is described below, in the Examples. In another embodiment, the polypeptide set forth in SEQ ID NO:9 is truncated immediately after the valine residue at position 507 of SEQ ID NO:9, set forth herein as SEQ ID NO:21. The activity of this molecule is described below, in the Examples.

In another embodiment, the internal region of amino acid residues from the leucine at position 147 of SEQ ID NO:9 to the methionine at position 425 of SEQ ID NO:9, inclusive, is deleted. This molecule is set forth herein as SEQ ID NO:23. The activity of this molecule is described below in the Examples. In another embodiment, the internal region of amino acid residues from the glutamine at position 218 of SEQ ID NO:9 to the alanine at position 392 of SEQ ID NO:9, inclusive, is deleted. This molecule is set forth herein as SEQ ID NO:25. The activity of this molecule is described below, in the Examples.

The invention also provides fragments with immunogenic properties. These contain an epitope-bearing portion of the subtilase-like protein and variants. These epitope-bearing peptides are useful to raise antibodies that bind specifically to a subtilase-like polypeptide or region or fragment. These peptides can contain at least 10, 12, at least 14, or between at least about 15 to about 30 amino acids.

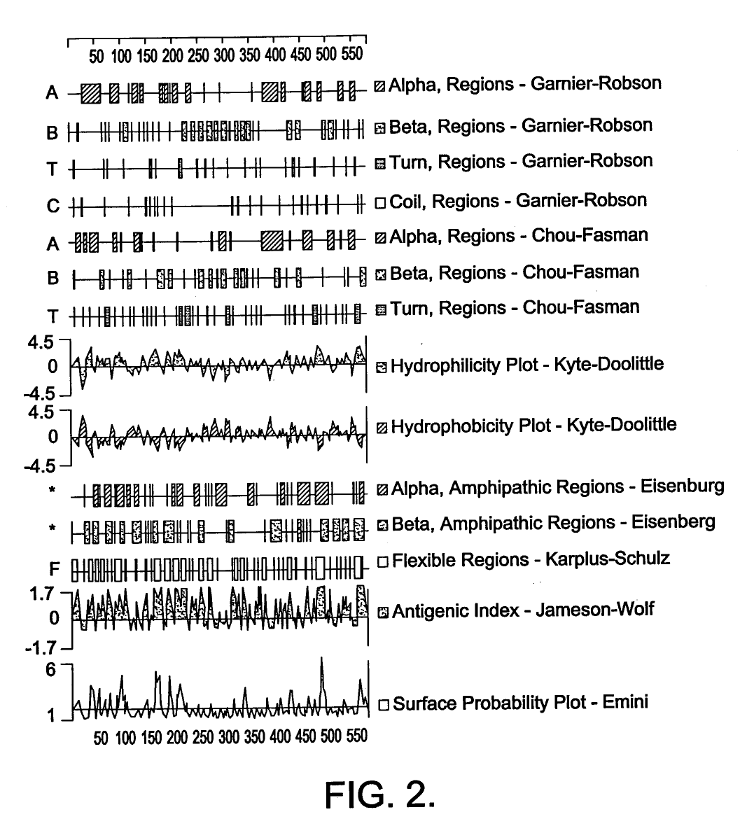

Non-limiting examples of antigenic polypeptides that can be used to generate antibodies include but are not limited to peptides derived from an extracellular site. Regions having a high antigenicity index are shown in FIG. 2. However, intracellularly-made antibodies (“intrabodies”) are also encompassed, which would recognize intracellular peptide regions.

The epitope-bearing subtilase-like polypeptides may be produced by any conventional means (Houghten, R. A. (1985) Proc. Natl. Acad. Sci. USA 82:5131-5135). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the subtilase-like peptide fragment and an additional region fused to the carboxyl terminus of the fragment.

The invention thus provides chimeric or fusion proteins. These comprise a subtilase-like peptide sequence operatively linked to a heterologous peptide having an amino acid sequence not substantially homologous to the subtilase-like protein. “Operatively linked” indicates that the subtilase-like peptide and the heterologous peptide are fused in-frame. The heterologous peptide can be fused to the N-terminus or C-terminus of the subtilase-like protein or can be internally located.

In one embodiment the fusion protein does not affect the subtilase-like protein function per se. For example, the fusion protein can be a GST-fusion protein in which the subtilase-like protein sequences are fused to the C-terminus of the GST sequences. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of recombinant subtilase-like protein. In certain host cells (e.g., mammalian host cells), expression and/or secretion of a protein can be increased by using a heterologous signal sequence. Therefore, in another embodiment, the fusion protein contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al. (1995) J. Mol. Recog. 8:52-58 (1995) and Johanson et al. J. Biol. Chem. 270:9459-9471). Thus, this invention also encompasses soluble fusion proteins containing a subtilase-like polypeptide and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence, which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al. (1992) Current Protocols in Molecular Biology). Moreover, many expression vectors are commercially available that already encode a fusion moiety (e.g., a GST protein). A subtilase-like protein-encoding nucleic acid can be cloned into such an expression vector such that the fusion moiety is linked in-frame to the subtilase-like protein.

Another form of fusion protein is one that directly affects the functions of the protein of the invention. Accordingly, a subtilase-like polypeptide is encompassed by the present invention in which one or more of the native protein domains (or parts thereof) has been replaced by homologous domains (or parts thereof) from another subtilase or subtilase-like protein. Accordingly, various permutations are possible. For example, the propeptide or subregion thereof can be replaced with the propeptide or subregion thereof from another subtilase or subtilase-like protein. As a further example, the catalytic domain or subregions thereof can be replaced; the P domain or subregion thereof can be replaced; the carboxyterminal region or parts thereof can be replaced; the transmembrane domain or parts thereof can be replaced; furthermore, domains not present in the native molecule could be added. These might include a cysteine-rich region, transmembrane region, or other carboxyterminal region if not present in the subtilase-like protein of the invention. Thus, chimeric proteins can be formed in which one or more of the native domains or subregions has been replaced by another.

Additionally, chimeric proteins can be produced in which one or more functional sites is derived from a different isoform, or from another subtilase or subtilase-like protein. It is understood however that sites could be derived from subtilases or subtilase-like proteins that occur in the mammalian genome but which have not yet been discovered or characterized. Such sites include, but are not limited to, those discussed above that affect such functions as autoproteolysis, substrate processing, secretion, subcellular localization, and specific membrane association, such as with the plasma membrane.

The isolated subtilase-like protein can be purified from cells that naturally express it, such as liver and testes, especially purified from cells that have been altered to express it (recombinant), or synthesized using known protein synthesis methods.

In one embodiment, the protein is produced by recombinant DNA techniques. For example, a nucleic acid molecule encoding the subtilase-like polypeptide is cloned into an expression vector, the expression vector introduced into a host cell and the protein expressed in the host cell. The protein can then be isolated from the cells by an appropriate purification scheme using standard protein purification techniques.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a proprotein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd ed., T. E. Creighton, W. H. Freeman and Company, New York, 1993. Many detailed reviews are available on this subject, such as by Wold, F., Posttranslational Covalent Modification of Proteins, B. C. Johnson, Ed., Academic Press, New York 1-12, 1983; Seifter et al. (1990) Meth. Enzymol. 182: 626-646 and Rattan et al. (1992) Ann. N.Y. Acad. Sci. 663:48-62.

As is also well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Polypeptide Uses

Cell-based and cell-free assays directed to expression or function of the NARC subtilase-like proteins are applicable to the uses disclosed herein. Cell-free assays include but are not limited to cleavage of substrate precursors and analogs, for example as disclosed in Nakayama et al., above. Cellular assays include recombinant cells coexpressing substrate precursor and the subtilase protein, such as disclosed in Nakayama et al., above. See also the assays disclosed in Wise et al. (1990) Proc. Natl. Acad. Sci. USA 87:9378-9382, Bresnahan et al. (1990) J. Cell Biol. 111:2851-2859, Van de Ven et al. (1990) Mol. Biol. Rep. 14:265-275, and Misumi et al. (1991) J. Biol. Chem. 266:16954-16959. Assays related to cellular toxin sensitivity include assays in RPE 40 cells, for example, as in Nakayama et al., above. Assays for pathogenic virulence can also be performed in transgenic animals as disclosed in Nakayama et al., above. Coexpression of precursor substrates and subtilases are also disclosed in Creemers et al., above and in Jutras et al. (1997) J. Biol. Chem. 272:15184-15188. Recombinant production of subtilases is disclosed in Seidah et al., above, and also in references cited therein (21, 30, 35-40). Moreover, recombinant production in milk of subtilase enzymes is disclosed in Seidah et al., above, and also in Lamango et al. (1996) Arch. Biochem. Biophys. 330:238-250. Coexpression of substrate precursors and subtilase enzymes are also disclosed in Seidah et al., above, and in references cited therein (35, 36, 37 and 42). Further, transgenic coexpression is also disclosed in Seidah et al., above, in Velander et al. (1997) Scientific American 276:70-74 and in Subramanian et al. (1996) Ann. NY Acad. Sci. 782:87-96. All of these references are incorporated herein by reference for disclosure of these assays. It is also understood that these assays apply not only to polypeptide uses but also to uses of any of the nucleic acids or antibodies disclosed herein.

Further, apoptosis-specific assays may be used to identify modulators of any of the target nucleic acids or proteins of the present invention, which proteins and/or nucleic acids are related to apoptosis. Accordingly, an agent that modulates the level or activity of any of these nucleic acids or proteins can be identified by means of apoptosis-specific assays. For example, high throughput screens exist to identify apoptotic cells by the use of chromatin or cytoplasmic-specific dyes. Thus, hallmarks of apoptosis, cytoplasmic condensation and chromosome fragmentation, can be used as a marker to identify modulators of any of the genes related to programmed-cell death described herein. Other assays include, but are not limited to, the activation of specific endogenous proteases, loss of mitochondrial function, cytoskeletal disruption, cell shrinkage, membrane blebbing, and nuclear condensation due to degradation of DNA.

Apoptosis can be actively induced in animal cells by a diverse array of triggers that range from ionizing radiation to hypothermia to viral infections to immune reactions. Majno et al. (1995) Amer. J. Pathol. 146:3-15; Hockenberry et al. (1995) Bio Essays 17:631-638; Thompson et al. (1995) Science 267:1456-1462.

Apoptosis can be triggered by the addition of apoptosis-promoting ligands to a cell in culture or in vivo. Apoptosis can also be triggered by decreasing or removing an apoptosis-inhibiting or survival-promoting ligand. Accordingly, apoptosis is triggered in view of the fact that the cell lacks a signal from a cell surface survival factor receptor. Ligands include, but are not limited to, FasL. Death-inhibiting ligands include, but are not limited to, IL-2. See Hetts et al. (1998) JAMA 279:300-307 (incorporated by reference in its entirety for teaching of ligands involved in active and passive apoptosis pathways). Central in the pathway, and also serving as potential molecules for inducing (or releasing from inhibition) apoptosis pathways include FADD, caspases, human CED4 homolog (also called apoptotic protease activating factor 1), the Bcl-2 family of genes including, but not limited to, apoptosis promoting (for example, Bax and Bad) and apoptosis inhibiting (for example, Bcl-2 and Bcl-x₁) molecules. See Hetts et al., above.

Multiple caspases upstream of caspase-3 can be inhibited by viral proteins such as cowpox, CrmA, and baculovirus, p35. Synthetic tripeptides and tetrapeptides inhibit casepase-3 specifically (Hetts, above).

Accordingly, cellular and animal models also exist for studying expression or function of the subtilase-like protein sequences in apoptosis and with regard to their effect on apoptosis. Such model systems can be applied in the context of the assays described herein below, for example the effect of specific mutations in the subtilase-like protein, the effect of compounds on the subtilase-like protein, and any of the other assays in which the effect of altered expression or activity of the subtilase-like protein is within the context of effects on apoptosis.

The protein sequences of the present invention can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The subtilase-like polypeptides are useful for producing antibodies specific for the subtilase-like protein, regions, or fragments. Regions having a high antigenicity index score are shown in FIG. 2.

A polypeptide and fragments and sequences thereof and antibodies specific thereto can be used to map the location of the gene encoding the polypeptide on a chromosome. This mapping can be carried out by specifically detecting the presence of the polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:34-40. Alternatively, the presence of the polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

The subtilase-like polypeptides are useful for providing desired amounts, including commercially valuable amounts, of a mature protein from a proprotein precursor. Accordingly, in one embodiment the present invention is valuable in that large amounts of a precursor protein can be produced in a recombinant cell in which the subtilase-like protein of the present invention is also overexpressed. This allows for the production of relatively large amounts of a mature protein produced by subtilase cleavage.

The subtilase-like polypeptides are also useful for producing reagents that inhibit viral or bacterial infection. Accordingly, the production of a propeptide that acts as a potent competitive inhibitor of the natural subtilase-like protein can be used to prevent the processing of bacterial endotoxins and viral envelope glycoproteins and hence to prevent infection.

The subtilase-like polypeptides of the invention are also useful as a screen for developing inhibitors of protein activation. Such inhibitors are useful, among other uses, for preventing pathogenic infection. In this regard, the polypeptides are useful in drug screening assays as described further herein below.

The subtilase-like polypeptides are useful for biological assays related to the subtilase-like proteins. Such assays involve any of the known subtilase functions or activities or properties useful for diagnosis and treatment of subtilase-like protein-related conditions, such as those disclosed herein.

The subtilase-like polypeptides are also useful in drug screening assays, in cell-based or cell-free systems. Cell-based systems can be native, i.e., cells that normally express the subtilase-like protein, as a biopsy or expanded in cell culture. In one embodiment, however, cell-based assays involve recombinant host cells expressing the subtilase-like protein.

Determining the ability of the test compound to interact with the subtilase-like protein can also comprise determining the ability of the test compound to preferentially bind to the polypeptide as compared to the ability of a known binding molecule to bind to the polypeptide. Such molecules include but are not limited to glycosylation enzymes, phosphorylation enzymes such as casein kinase II, substrate precursor proteins, cleaved propeptides, and membrane components, for example those that interact with a transmembrane domain. Substrates include any of those disclosed herein known to be processed by subtilases, that include but are not limited to growth factors and hormones, including mouse pro-β-nerve growth factor, porcine pro-brain-derived neurotrophic factor, human pro-neurotrophin-3, human pro-transforming growth factor β1, rat pro-Müllerian inhibiting substance, human pro-insulin-like growth factor I, human pro-endothelin-1, human pro-parathyroid hormone-related peptide, human pro-parathyroid hormone; receptors, including human insulin pro-receptor, human hepatocyte growth factor pro-receptor, human pro-LRP, human integrin α3-chain, human integrin α6-chain; plasma proteins, including human proalbumin, rat complement pro-C3, human pro-factor IX, human pro-factor X, human pro-von Willebrand Factor, human proprotein C; matrix metalloproteinases, including human stromelysin-3, human MT-MMP 1; viral envelope glycoproteins, including human immunodeficiency virus gp160, human cytomegalovirus glycoprotein B, mouse mammary tumor virus-7 superantigen, avian influenza virus A hemagglutinin, measles virus F₀, Newcastle disease virus F₀, Sindbis virus gpE2, human parainfluenza virus type 3 F₀; bacterial exotoxins, including anthrax toxin protective antigen, diphtheria toxin, Pseudomonas exotoxin A, Shiga toxin; and others, including human pro-furin, rat pro-endopeptidase 3.4.24. and 18, and mouse pro-7B2.

The polypeptides can be used to identify compounds that can modulate the subtilase-like protein activity. Such compounds, for example, can increase or decrease affinity or rate of binding to substrate, compete with substrate for binding to the protein, or displace substrate bound to the protein. Both subtilase-like protein and appropriate variants and fragments can be used in high-throughput screens to assay candidate compounds for the ability to bind to the protein. These compounds can be further screened against a functional subtilase-like protein to determine the effect of the compound on the protein activity. Compounds can be identified that activate (agonist) or inactivate (antagonist) the protein to a desired degree. Modulatory methods can be performed in vitro (e.g., by culturing the cell with the agent) or, alternatively, in vivo (e.g., by administering the agent to a subject.

The subtilase-like polypeptides can be used to screen a compound for the ability to stimulate or inhibit interaction between the protein and a target molecule that normally interacts with the protein. The target can be any of the molecules with which the protein interacts as described herein. The assay includes the steps of combining the protein with a candidate compound under conditions that allow the protein or fragment to interact with the target molecule, and to detect the formation of a complex between the protein and the target or to detect the biochemical consequence of the interaction with the protein and the target. Such consequences include production of a mature substrate molecule, for example mature insulin from pro-insulin, or include the biological consequences of cleavage (or lack thereof), such as effects on embryogenesis, formation of extracellular matrix, pathogen virulence, cell proliferation, inflammation, apoptosis, blood clotting and complement function, cellular differentiation, metabolic activity, cell adhesion, cell signaling, and tumor formation. Moreover, such end results can also be assayed at the level of the organism to further include symptoms such as obesity, tumor formation, endocrine disorders, embryonic induction, bleeding time, and other effects of abnormal processing, including but not limited to those abnormal processing events disclosed herein.

Determining the ability of the subtilase-like protein to bind to a target molecule can also be accomplished using a technology such as real-time Bimolecular Interaction Analysis (BIA). Sjolander et al. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 97:6378-6382); (Felici (1991) J. Mol. Biol. 222:301-310); (Ladner supra).

Candidate compounds include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble full-length subtilase-like protein or mature fragment that competes for substrate binding. Other candidate compounds include mutant subtilase-like proteins or appropriate fragments containing mutations that affect the protein function and thus compete for substrate. Accordingly, a fragment that competes for substrate, for example with a higher affinity, or a fragment that binds substrate but does not degrade it, is encompassed by the invention.

Another candidate compound is a propeptide that acts as a competitive inhibitor of the mature subtilase-like protein.

WO 98/37910 discloses peptide inhibitors of propeptide/prohormone convertases. These inhibitors can be used to inhibit propeptide/prohormone convertases and to treat such disorders as cancer, endocrine disorders, and viral infections, including AIDS. Accordingly, the disclosure provides various peptides useful for inhibition, and longer peptides containing those peptides. The disclosure of those peptide sequences is incorporated herein by reference. In particular, see pages 6-16 or the sequence listing in the disclosure. With respect to the present invention, accordingly, such inhibitors are useful for treating the disorders, such as those disclosed herein, by inhibiting the subtilase-like protein of the present invention. Inhibition of conversion has uses that include, but are not limited to, reducing malignant transformation and tumorigenesis, reducing the physiological consequences of tumor production and release of bioactive peptides, such as those derived from insulinomas, gastrinomas, or lung cancer cells that may hypersecrete hormonally active peptides, inhibiting neoplasia by blocking subtilase-like protein-mediated processing of growth factors that are produced in many types of tumor cells, reducing or preventing HIV infection via inhibition of processing of gp160, thereby blocking formation of gp120, and diminishing the infectivity of newly synthesized virions. Inhibition of conversion is also useful for inhibiting overproduction of endocrine or neuroendocrine hormones that result in pathophysiology.

Other inhibitors include acylated peptidyl chloromethanes containing a consensus furin cleavage sequence, such as decanoyl-ARG-GLU-LYS-ARG-CH₂Cl. See, for example Stieneke-Grober (1992) EMBO Journal 11:2407-2414. Further candidates include reversible peptide inhibitors in which the —NH-group of the scissile P₁-P₁′ bond has been replaced with a methylene group or a methylene group has been inserted between the —CO— and —NH— of the scissile bond. See Angliker (1995) J. Med. Chem. 38:4014-4018. Protein-based furin inhibitors have also been developed, such as a variant of α₁-antitrypsin that has a replacement of the reactive-site MET residue by ARG. This has been shown to inhibit the in vitro conversion of proalbumin. See Bathurst (1987) Science 235:348-350. Other α₁-antitrypsin variants have been constructed, such as α₁-PDX in which the reactive center ALA^{P4}-ILE-PRO-MET^{P1} sequence has been replaced by ARG-ILE-PRO-ARG. See Mizuno et al. (1988) Biochem. Biophys. Resp. Commun. 156:246-254. This particular candidate has been shown to inhibit the cleavage of viral envelope glycoproteins, including HIV gp160.

The invention provides other end points to identify compounds that modulate (stimulate or inhibit) the subtilase-like protein activity. The assays typically involve an assay of molecular, subcellular, cellular, or in vivo events that indicate the subtilase-like protein activity. These include but are not limited to those that have been discussed above, including the production of mature substrate peptide, association with specific subcellular locations, effects on cell growth or differentiation, including apoptosis, pathogen virulence, obesity, and the like.

Thus, the expression of genes that are up- or down-regulated in response to the subtilase-like protein activity pathway can be assayed. In one embodiment, the regulatory region of such genes can be operably linked to a marker that is easily detectable, such as luciferase. Alternatively, phosphorylation of the subtilase-like protein or target could also be measured.

Any of the biological or biochemical functions mediated by the subtilase-like protein can be used as an endpoint assay. These include all of the biochemical or biochemical/biological events described herein, in the references cited herein, incorporated by reference for these endpoint assay targets, and other functions known to those of ordinary skill in the art.

Binding and/or activating compounds can also be screened by using chimeric subtilase-like proteins in which one or more domains, sites, and the like, as disclosed herein, or parts thereof, can be replaced by their heterologous counterparts derived from other subtilases or subtilase-like proteins or from other subtilase or subtilase-like isoforms. For example, a catalytic region can be used that interacts with a different substrate specificity and/or affinity than the native subtilase-like protein of the invention. Accordingly, a different set of components is available as an end-point assay for activation. Alternatively, a heterologous COOH sequence can replace a native COOH sequence or can be added where no COOH sequence existed. This will result in different subcellular or cellular localization and accordingly can result in having an effect on a different set of components or pathway. Accordingly, a different set of components or pathway is available as an endpoint assay for activation. As a further alternative, the site of modification by an effector protein, for example phosphorylation by casein kinase II, can be replaced with the site from a different effector protein. Activation can also be detected by a reporter gene containing an easily detectable coding region operably linked to a transcriptional regulatory sequence that is part of the native signal transduction pathway.

The subtilase-like polypeptides are also useful in competition binding assays in methods designed to discover compounds that interact with the polypeptide. Thus, a compound is exposed to the polypeptide under conditions that allow the compound to bind or to otherwise interact with the polypeptide. Soluble subtilase-like polypeptide is also added to the mixture. If the test compound interacts with the soluble subtilase-like polypeptide, it decreases the amount of complex formed with or activity from the subtilase-like polypeptide target. This type of assay is particularly useful in cases in which compounds are sought that interact with specific regions of the subtilase-like protein. Thus, the soluble polypeptide that competes with the target protein region is designed to contain peptide sequences corresponding to the region of interest.

Another type of competition-binding assay can be used to discover compounds that interact with specific functional sites. As an example, substrate and a candidate compound can be added to a sample of the subtilase-like protein. Compounds that interact with the subtilase-like protein at the same site as the substrate will reduce the amount of complex formed between the subtilase-like protein and substrate. Accordingly, it is possible to discover a compound that specifically prevents or alters interaction between the subtilase-like protein and substrate. Another example involves adding a candidate compound to a sample of subtilase-like protein and propeptide. A compound that competes with the propeptide will reduce the amount of binding of the propeptide to the subtilase-like protein. Accordingly, compounds can be discovered that directly interact with the subtilase-like protein and compete with the propeptide. Such assays can involve any other component that interacts with the subtilase-like protein.

To perform cell free drug screening assays, it is desirable to immobilize either the subtilase-like protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/subtilase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes is dissociated. Alternatively, the complexes can be dissociated from the matrix, separated by SDS-PAGE, and the level of subtilase-like-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a subtilase-like-binding target component and a candidate compound are incubated in the subtilase-like protein-presenting wells and the amount of complex trapped in the well can be quantitated. Methods for detecting such complexes, in addition to those described above for the GST-immobilized complexes, include immunodetection of complexes using antibodies reactive with the subtilase-like target molecule, or which are reactive with the subtilase-like protein and compete with the target molecule; as well as enzyme-linked assays which rely on detecting an enzymatic activity associated with the target molecule.

Modulators of the subtilase-like protein activity identified according to these drug screening assays can be used to treat a subject with a disorder mediated by the subtilase-like protein pathway, by treating cells that express the subtilase-like protein. These methods of treatment include the steps of administering the modulators of the subtilase-like protein activity in a pharmaceutical composition as described herein, to a subject in need of such treatment.

Treatment is of disorders related to improper proprotein processing. Disorders result from events including but not limited to processing of the extracellular matrix, growth factors, including during early embryogenesis, serum proteins, including proteases of blood clotting and complement systems, matrix metalloproteinases, receptors, enzymes, adhesion molecules, hormones, cell surface signaling components, and endocrine and neural polypeptide hormones. Accordingly, treatment is of the consequences of such abnormal processing of these components, including defects in embryogenesis, tumor formation, inflammation, apoptosis, defects in differentiation, improper metabolic activity, defects in cell signaling, defects in programmed cell death, and endocrine disorders and endocrine tumors resulting from improper prohormone processing. On another level, treatment can be of such disorders as obesity. Further, since pathogenic virulence is related to processing, disorders also include increased virulence as a result of over-expression or increased activity of the subtilase-like protein, resulting in relatively high viral and bacterial virulence.

In the present case, a relevant disorder that maps to chromosome 1 p34-p32 is the muscle-eye brain disease (MEB). See www.ncbi.nlm.nih.gov/htbin-post/Omim/dispmim253280. This text describes a disorder comprising congenital muscular dystrophy with high serum CPK, severe congenital myopia, congenital glaucoma, pallor of the optic discs, retinal hypoplasia, mental retardation, hydrocephalus, abnormal EEG, and myoclonic jerks. Characteristics are severe early-onset muscle weakness, mental retardation and pathologic eye findings, usually congenital myopia. A further study showed the combination of congenital muscular dystrophy and involvement of the central nervous system and eyes. This disease has phenotypic similarities with the Walker-Warburg syndrome.

A further relevant disorder is described in www.ncbi.nlm.nih.gov/htbin-post/Omim/dispmim602522, designated Bartter Syndrome, infantile, with sensorineural deafness. Bartter syndrome is an autosomal recessive disorder defined by hypokalemic metabolic alkalosis. Affected individuals have elevated plasma renin activity and hyperaldosteronism, with normal blood pressure, altered prostaglandin metabolism (with increased levels of urinary prostaglandins), and increased urinary chloride excretion. One form of Bartter syndrome is due to mutation in the kidney chloride channel B and maps to 1p36.

Other especially relevant disorders include those that are associated with programmed cell death, and particularly with neuronal programmed cell death. These include but are not limited to those described herein and also in the cross-referenced applications above, that are incorporated herein by reference for disclosure of disorders associated with neuronal programmed cell death.

As used herein, “programmed cell death” refers to a genetically regulated process involved in the normal development of multicellular organisms. This process occurs in cells destined for removal in a variety of normal situations, including larval development of the nematode C. elegans, insect metamorphosis, development in mammalian embryos, including the nephrogenic zone in the developing kidney, and regression or atrophy (e.g., in the prostate after castration). Programmed cell death can occur following the withdrawal of growth and trophic factors in many cells, nutritional deprivation, hormone treatment, ultraviolet irradiation, and exposure to toxic and infectious agents including reactive oxygen species and phosphatase inhibitors, e.g., okadaic acid, calcium ionophores, and a number of cancer chemotherapeutic agents. See Wilson (1998) Biochem. Cell Biol. 76:573-582 and Hetts (1998) JAMA 279:300-307, the contents of which are incorporated herein by reference. Thus, the proteins of the invention, by being differentially expressed during programmed cell death, e.g., neuronal programmed cell death, can modulate a programmed cell death pathway activity and provide novel diagnostic targets and therapeutic agents for disorders characterized by deregulated programmed cell death, particularly in cells that express the protein.

As used herein, a “disorder characterized by deregulated programmed cell death” refers to a disorder, disease or condition which is characterized by a deregulation, e.g., an upregulation or a downregulation, of programmed cell death. Programmed cell death deregulation can lead to deregulation of cellular proliferation and/or cell cycle progression. Examples of disorders characterized by deregulated programmed cell death include, but are not limited to, neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementias; myelodysplastic syndromes, e.g., aplastic anemia; ischemic injury, e.g., myocardial infarction, stroke, or reperfusion injury; autoimmune disorders, e.g., systemic lupus erythematosus, or immune-mediated glomerulonephritis; or profilerative disorders, e.g., cancer, such as follicular lymphomas, carcinomas with p53 mutations, or hormone-dependent tumors, e.g., breast cancer, prostate cancer, or ovarian cancer). Clinical manifestations of faulty apoptosis are also seen in stroke and in rheumatoid arthritis. Wilson (1998) Biochem. Cell. Biol. 76:573-582.

Failure to remove autoimmune cells that arise during development or that develop as a result of somatic mutation during an immune response can result in autoimmune disease. One of the molecules that plays a critical role in regulating cell death in lymphocytes is the cell surface receptor for Fas.

Viral infections, such as those caused by herpesviruses, poxyiruses, and adenoviruses, may result in aberrant apoptosis. Populations of cells are often depleted in the event of viral infection, with perhaps the most dramatic example being the cell depletion caused by the human immunodeficiency virus (HIV). Most T cells that die during HIV infections do not appear to be infected with HIV. Stimulation of the CD4 receptor may result in the enhanced susceptibility of uninfected T cells to undergo apoptosis.

Many disorders can be classified based on whether they are associated with abnormally high or abnormally low apoptosis. Thompson (1995) Science 267:1456-1462. Apoptosis may be involved in acute trauma, myocardial infarction, stroke, and infectious diseases, such as viral hepatitis and acquired immunodeficiency syndrome.

Primary apoptosis deficiencies include graft rejection. Accordingly, the invention is relevant to the identification of genes useful in inhibiting graft rejection.

Primary apoptosis deficiencies also include autoimmune diabetes. Accordingly, the invention is relevant to the identification of genes involved in autoimmune diabetes and accordingly, to the identification of agents that act on these targets to modulate the expression of these genes and hence, to treat or diagnose this disorder. Further, it has been suggested that all autoimmune disorders can be viewed as primary deficiencies of apoptosis (Hetts, above). Accordingly, the invention is relevant for screening for gene expression and transcriptional profiling in any autoimmune disorder and for screening for agents that affect the expression or transcriptional profile of these genes.

Primary apoptosis deficiencies also include local self reactive disorder. This includes Hashimoto thyroiditis.

Primary apoptosis deficiencies also include lymphoproliferation and autoimmunity. This includes, but is not limited to, Canale-Smith syndrome.

Primary apoptosis deficiencies also include cancer. For example, p53 induces apoptosis by acting as a transcription factor that activates expression of various apoptosis-mediating genes or by upregulating apoptosis-mediating genes such as Bax.

Primary apoptosis excesses are associated with neurodegenerative disorders including Alzheimer's disease, Parkinson's disease, spinal muscular atrophy, and amyotrophic lateral sclerosis.

Primary apoptosis excesses are also associated with heart disease including idiopathic dilated cardiomyopathy, ischemic cardiomyopathy, and valvular heart disease. Evidence has also been shown of apoptosis in heart failure resulting from arrhythmogenic right ventricular dysplasia. For all these disorders, see Hetts, above.

Death receptors also include the TNF receptor-1 and hence, TNF acts as a death ligand.

A wide variety of neurological diseases are characterized by the gradual loss of specific sets of neurons. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow.

These disorders could result from the activation of genes that promote apoptosis, acquired deficiencies in stromal cells or hematopoietic survival factors, or the direct effects of toxins and mediators of immune responses.

Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

The invention also pertains to disorders of the central nervous system (CNS). These disorders include, but are not limited to cognitive and neurodegenerative disorders such as Alzheimer's disease, senile dementia, Huntington's disease, amyotrophic lateral sclerosis, and Parkinson's disease, as well as Gilles de la Tourette's syndrome, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II). Further CNS-related disorders include, for example, those listed in the American Psychiatric Association's Diagnostic and Statistical manual of Mental Disorders (DSM), the most current version of which is incorporated herein by reference in its entirety.

As used herein, “differential expression” or differentially expressed” includes both quantative and qualitative differences in the temporal and/or cellular expression pattern of a gene, e.g., the programmed cell death genes disclosed herein, among, for example, normal cells and cells undergoing programmed cell death. Genes which are differentially expressed can be used as part of a prognostic or diagnostic marker for the evaluation of subjects at risk for developing a disorder characterized by deregulated programmed cell death. Depending on the expression level of the gene, the progression state of the disorder can also be evaluated.

Further relevant disorders include those associated with aberrant mitochondrial function. Open reading frame analysis of the human subtilase-like protein of the present invention indicates that the enzyme is localized in mitochondria.

The yeast V-ATPase is similar to the V-ATPases of higher organisms and has shown to be an accessible model for many aspects of V-ATPase function. See Kane, J. (1999) Bioenergetics Biomembranes 31:40-56. In yeast this ATPase acidifies the vacuole to a pH of approximately 6 and drives secondary transport of calcium, amino acids and other nutrients. V-ATPases also reside in other intracellular compartments. Accordingly, the yeast ATPase is analogous to the role of V-ATPases in intracellular compartments of all eukaryotic cells. The V-ATPase is a substrate for subtilase-related enzymes. Accordingly, with regard to the present invention, further relevant disorders include those that result from defective V-ATPase processing.

Since the gene is expressed in (among others) liver, kidney, and testes, further relevant disorders are those involving these tissues, especially liver, where the gene is relatively highly expressed, and particularly apoptosis-related liver disorders.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, α₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, Leydig (interstitial) cell tumors and sertoli cell tumors (androblastoma), and testicular lymphoma, and miscellaneous lesions of tunica vaginalis.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

The subtilase-like polypeptides are thus useful for treating a subtilase-like protein-associated disorder characterized by aberrant expression or activity of the subtilase-like protein. In one embodiment, the method involves administering an agent (e.g., an agent identified by a screening assay described herein), or combination of agents that modulates (e.g., upregulates or downregulates) expression or activity of the protein. In another embodiment, the method involves administering the protein as therapy to compensate for reduced or aberrant expression or activity of the protein.

Methods for treatment include but are not limited to the use of soluble subtilase-like protein or fragments of the subtilase-like protein that compete for substrate or propeptide. These proteins or fragments can have a higher affinity for the target so as to provide effective competition. Methods of treatment also include the use of candidate compounds as described hereinabove.

Stimulation of activity is desirable in situations in which the protein is abnormally downregulated and/or in which increased activity is likely to have a beneficial effect. Likewise, inhibition of activity is desirable in situations in which the protein is abnormally upregulated and/or in which decreased activity is likely to have a beneficial effect. In one example of such a situation, a subject has a disorder characterized by aberrant development or cellular differentiation. In another example, the subject has a proliferative disease (e.g., cancer) or a disorder characterized by an aberrant hematopoietic response. In another example, it is desirable to achieve tissue regeneration in a subject (e.g., where a subject has undergone brain or spinal cord injury and it is desirable to regenerate neuronal tissue in a regulated manner).

In yet another aspect of the invention, the proteins of the invention can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins (captured proteins) which bind to or interact with the proteins of the invention and modulate their activity.

The subtilase-like polypeptides also are useful to provide a target for diagnosing a disease or predisposition to disease mediated by the subtilase-like protein, and particularly in obesity, liver disorders, and disorders related to neuronal programmed cell death. Accordingly, methods are provided for detecting the presence, or levels of, the subtilase-like protein in a cell, tissue, or organism. The method involves contacting a biological sample with a compound capable of interacting with the protein such that the interaction can be detected.

One agent for detecting the protein is an antibody capable of selectively binding to the protein. A biological sample includes tissues, cells and biological fluids isolated from a subject, as well as tissues, cells and fluids present within a subject.

The subtilase-like protein also provides a target for diagnosing active disease, or predisposition to disease, in a patient having a variant of the subtilase-like protein. Thus, the subtilase-like protein can be isolated from a biological sample and assayed for the presence of a genetic mutation that results in an aberrant protein. This includes amino acid substitution, deletion, insertion, rearrangement, (as the result of aberrant splicing events), and inappropriate post-translational modification. Analytic methods include altered electrophoretic mobility, altered tryptic peptide digest, altered protein activity in cell-based or cell-free assay, alteration in substrate binding or degradation, propeptide binding or phosphorylation, or antibody-binding pattern, altered isoelectric point, direct amino acid sequencing, and any other of the known assay techniques useful for detecting mutations in a protein in general or in a subtilase-like protein specifically.

In vitro techniques for detection of the subtilase-like protein include enzyme linked immunosorbent assays (ELISAs), Western blots, immunoprecipitations and immunofluorescence. Alternatively, the protein can be detected in vivo in a subject by introducing into the subject a labeled antibody. For example, the antibody can be labeled with a radioactive marker whose presence and location in a subject can be detected by standard imaging techniques. Particularly useful are methods, which detect the allelic variant of the subtilase-like protein expressed in a subject, and methods, which detect fragments of the protein in a sample.

The subtilase-like polypeptides are also useful in pharmacogenomic analysis. Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11):983-985, and Linder, M. W. (1997) Clin. Chem. 43(2):254-266. The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes affects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants of the subtilase-like protein in which one or more of the protein functions in one population is different from those in another population. The polypeptides thus allow a target to ascertain a genetic predisposition that can affect treatment modality. Thus, in a substrate (analog) based treatment, polymorphism may give rise to catalytic regions that are more or less active. Accordingly, dosage would necessarily be modified to maximize the therapeutic effect within a given population containing the polymorphism. As an alternative to genotyping, specific polymorphic polypeptides could be identified.

The subtilase-like polypeptides are also useful for monitoring therapeutic effects during clinical trials and other treatment. Thus, the therapeutic effectiveness of an agent that is designed to increase or decrease gene expression, protein levels or protein activity can be monitored over the course of treatment using the subtilase-like polypeptides as an end-point target. The monitoring can be, for example, as follows: (i) obtaining a pre-administration sample from a subject prior to administration of the agent; (ii) detecting the level of expression or activity of the protein in the pre-administration sample; (iii) obtaining one or more post-administration samples from the subject; (iv) detecting the level of expression or activity of the protein in the post-administration samples; (v) comparing the level of expression or activity of the protein in the pre-administration sample with the protein in the post-administration sample or samples; and (vi) increasing or decreasing the administration of the agent to the subject accordingly.

Antibodies

The invention also provides antibodies that selectively bind to the subtilase-like protein and its variants and fragments. An antibody is considered to selectively bind, even if it also binds to other proteins that are not substantially homologous with the subtilase-like protein. These other proteins share homology with a fragment or domain of the subtilase-like protein. This conservation in specific regions gives rise to antibodies that bind to both proteins by virtue of the homologous sequence. In this case, it would be understood that antibody binding to the subtilase-like protein is still selective.

To generate antibodies, an isolated subtilase-like polypeptide is used as an imnimunogen to generate antibodies using standard techniques for polyclonal and monoclonal antibody preparation. Either the full-length protein or antigenic peptide fragment can be used. Regions having a high antigenicity index are shown in FIG. 2.

Antibodies are preferably prepared from these regions or from discrete fragments in these regions. However, antibodies can be prepared from any region of the peptide as described herein. A preferred fragment produces an antibody that diminishes or completely prevents substrate hydrolysis or binding. Antibodies can be developed against the entire protein or domains of the protein as described herein. Antibodies can also be developed against specific functional sites as disclosed herein.

The antigenic peptide can comprise a contiguous sequence of at least 12, 14, 15, or 30 amino acid residues. In one embodiment, fragments correspond to regions that are located on the surface of the protein, e.g., hydrophilic regions. These fragments are not to be construed, however, as encompassing any fragments, which may be disclosed prior to the invention.

Antibodies can be polyclonal or monoclonal. An intact antibody, or a fragment thereof (e.g. Fab or F(ab′)₂) can be used.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An appropriate immunogenic preparation can be derived from native, recombinantly expressed, or chemically synthesized peptides.

Antibody Uses

The antibodies can be used to isolate the subtilase-like proteins of the invention by standard techniques, such as affinity chromatography or immunoprecipitation. The antibodies can facilitate the purification of the natural subtilase-like protein from cells and a recombinantly produced subtilase-like protein expressed in host cells.

The antibodies are useful to detect the presence of the subtilase-like protein in cells or tissues to determine the pattern of expression of the protein among various tissues in an organism and over the course of normal development.

The antibodies can be used to detect the subtilase-like protein in situ, in vitro, or in a cell lysate or supernatant in order to evaluate the abundance and pattern of expression.

The antibodies can be used to assess abnormal tissue distribution or abnormal expression during development.

Antibody detection of circulating fragments of the full length protein can be used to identify protein turnover.

Further, the antibodies can be used to assess the subtilase-like protein expression in disease states such as in active stages of the disease or in an individual with a predisposition toward disease related to the protein function. When a disorder is caused by an inappropriate tissue distribution, developmental expression, or level of expression of the protein, the antibody can be prepared against the normal protein. If a disorder is characterized by a specific mutation in the protein, antibodies specific for this mutant protein can be used to assay for the presence of the specific mutant protein. However, intracellularly-made antibodies (“intrabodies”) are also encompassed, which would recognize intracellular peptide regions in the protein.

The antibodies can also be used to assess normal and aberrant subcellular localization of cells in the various tissues in an organism. Antibodies can be developed against the whole protein or portions of the protein.

The diagnostic uses can be applied, not only in genetic testing, but also in monitoring a treatment modality. Accordingly, where treatment is ultimately aimed at correcting the subtilase-like protein expression level or the presence of aberrant proteins and aberrant tissue distribution or developmental expression, antibodies directed against the protein or relevant fragments can be used to monitor therapeutic efficacy.

Antibodies accordingly can be used diagnostically to monitor protein levels in tissue as part of a clinical testing procedure, e.g., to, for example, determine the efficacy of a given treatment regimen.

Additionally, antibodies are useful in pharmacogenomic analysis. Thus, antibodies prepared against a polymorphic subtilase-like protein can be used to identify individuals that require modified treatment modalities.

The antibodies are also useful as diagnostic tools as an immunological marker for aberrant subtilase-like protein analyzed by electrophoretic mobility, isoelectric point, tryptic peptide digest, and other physical assays known to those in the art.

The antibodies are also useful for tissue typing. Thus, where a specific subtilase-like protein has been correlated with expression in a specific tissue, antibodies that are specific for this subtilase-like protein can be used to identify a tissue type.

The antibodies are also useful in forensic identification. Accordingly, where an individual has been correlated with a specific genetic polymorphism resulting in a specific polymorphic protein, an antibody specific for the polymorphic protein can be used as an aid in identification.

The antibodies are also useful for inhibiting protein function, for example, blocking substrate, propeptide, or the subcellular localization site(s).

These uses can also be applied in a therapeutic context in which treatment involves inhibiting protein function. An antibody can be used, for example, to block substrate binding. Antibodies can be prepared against specific fragments containing sites required for function or against intact protein associated with a cell.

Completely human antibodies are particularly desirable for therapeutic treatment of human patients. For an overview of this technology for producing human antibodies, see Lonberg et al. (1995) Int. Rev. Immunol. 13:65-93. For a detailed discussion of this technology for producing human antibodies and human monoclonal antibodies and protocols for producing such antibodies, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

Another aspect of the invention relates to a method for inducing an immunological response in a mammal which comprises inoculating the mammal with a polypeptide of the present invention, adequate to produce antibody and/or T cell immune response to protect the animal from the diseases herein mentioned, among others. Yet another aspect of the invention relates to a method of inducing immunological response in a mammal which comprises delivering a polypeptide of the present invention via a vector directing expression of the polynucleotide and coding for the polypeptide in vivo in order to induce such an immunological response to produce antibody to protect the animal from diseases.

A further aspect of the invention relates to an immunological/vaccine formulation (composition) which, when introduced into a mammalian host, induces an immunological response in that mammal to a polypeptide of the present invention where the composition comprises a polypeptide or polynucleotide of the present invention. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain antioxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

The invention also encompasses kits for using antibodies to detect the presence of the subtilase-like protein in a biological sample. The kit can comprise antibodies such as a labeled or labelable antibody and a compound or agent for detecting the protein in a biological sample; means for determining the amount of protein in the sample; and means for comparing the amount of protein in the sample with a standard. The compound or agent can be packaged in a suitable container. The kit can further comprise instructions for using the kit to detect the protein.

Polynucleotides

The nucleotide sequences in SEQ ID NOS:2, 4, 6, and 8 were obtained by sequencing the human, mouse, and rat cDNA.

The specifically disclosed cDNAs comprise the coding region and 5′ and 3′ untranslated sequences in SEQ ID NOS:2, 4, 6, or 8.

The invention provides isolated polynucleotides encoding the novel subtilase-like proteins. The term “subtilase-like polynucleotide” or “subtilase-like nucleic acid” refers to the sequences shown in SEQ ID NOS:2, 4, 6, or 8. The term “subtilase-like polynucleotide” or “subtilase-like nucleic acid” further includes variants and fragments of the subtilase-like polynucleotides.

An “isolated” subtilase-like nucleic acid is one that is separated from other nucleic acid present in the natural source of the subtilase-like nucleic acid. Preferably, an “isolated” nucleic acid is free of sequences which naturally flank the subtilase-like nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the subtilase-like nucleic acid is isolated from flanking sequences such that it can be subjected to the specific manipulations described herein, such as recombinant expression, preparation of probes and primers, and other uses specific to the subtilase-like nucleic acid sequences.

Moreover, an “isolated” nucleic acid molecule, such as a cDNA or RNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

In some instances, the isolated material will form part of a composition (or example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

The subtilase-like polynucleotides can encode the mature protein plus additional amino or carboxyterminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The subtilase-like polynucleotides include, but are not limited to, the sequence encoding the mature polypeptide alone, the sequence encoding the mature polypeptide and additional coding sequences, such as a leader or secretory sequence (e.g., a pre-pro or proprotein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

Subtilase-like polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

Subtilase-like nucleic acid can comprise a nucleotide sequence shown in SEQ ID NOS:2, 4, 6, or 8, corresponding to human, mouse, or rat cDNA.

In one embodiment, the subtilase-like nucleic acid comprises only the coding region.

The invention further provides variant subtilase-like polynucleotides, and fragments thereof, that differ from a nucleotide sequence shown in SEQ ID NOS:2, 4, 6, or 8 due to degeneracy of the genetic code and thus encode the same protein as that encoded by the nucleotide sequences.

The invention also provides subtilase-like nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Typically, variants have a substantial identity with a nucleic acid molecule of SEQ ID NOS:2, 4, 6, or 8 and the complements thereof. Variation can occur in either or both the coding and non-coding regions. The variations can produce both conservative and non-conservative amino acid substitutions.

Orthologs, homologs, and allelic variants can be identified using methods well known in the art. These variants comprise a nucleotide sequence encoding a subtilase-like protein that is at least about 60-65%, 65-70%, typically at least about 70-75%, more typically at least about 80-85%, and most typically at least about 90-95% or more homologous to a nucleotide sequence shown in SEQ ID NOS:2, 4, 6, or 8 or a fragment of this sequence. Such nucleic acid molecules can readily be identified as being able to hybridize under stringent conditions, to a nucleotide sequence shown in SEQ ID NOS:2, 4, 6, or 8, or a fragment of the sequence. It is understood that stringent hybridization does not indicate substantial homology where it is due to general homology, such as poly A sequences, or sequences common to all or most proteins, all subtilases, or common to a known subtilase family. Moreover, it is understood that variants do not include any of the nucleic acid sequences that may have been disclosed prior to the invention.

As used herein, the term “hybridizes under stringent conditions” is intended to describe conditions for hybridization and washing under which nucleotide sequences encoding a polypeptide at least about 60-65% homologous to each other typically remain hybridized to each other. The conditions can be such that sequences at least about 65%, at least about 70%, at least about 75%, at least about 80%, at least about 90%, at least about 95% or more identical to each other remain hybridized to one another. Such stringent conditions are known to those skilled in the art and can be found in Current Protocols in Molecular Biology, John Wiley & Sons, N.Y. (1989), 6.3.1-6.3.6, incorporated by reference. One example of stringent hybridization conditions is hybridization in 6× sodium chloride/sodium citrate (SSC) at about 45° C., followed by one or more washes in 0.2× SSC, 0.1% SDS at 50-65° C. In another non-limiting example, nucleic acid molecules are allowed to hybridize in 6× sodium chloride/sodium citrate (SSC) at about 45° C., followed by one or more low stringency washes in 0.2× SSC/0.1% SDS at room temperature, or by one or more moderate stringency washes in 0.2× SSC/0.1% SDS at 42° C., or washed in 0.2× SSC/0.1% SDS at 65° C. for high stringency. In another embodiment, hybridization is in 3× SSC, at about 65° C., followed by washes that include 0.2× SSC at 65° C. for at least 30 minutes. In one embodiment, an isolated nucleic acid molecule that hybridizes under stringent conditions to a sequence of SEQ ID NOS:2, 4, 6, or 8 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

As understood by those of ordinary skill, the exact conditions can be determined empirically and depend on ionic strength, temperature and the concentration of destabilizing agents such as formamide or denaturing agents such as SDS. Other factors considered in determining the desired hybridization conditions include the length of the nucleic acid sequences, base composition, percent mismatch between the hybridizing sequences and the frequency of occurrence of subsets of the sequences within other non-identical sequences. Thus, equivalent conditions can be determined by varying one or more of these parameters while maintaining a similar degree of identity or similarity between the two nucleic acid molecules.

The present invention also provides isolated nucleic acids that contain a single or double stranded fragment or portion that hybridizes under stringent conditions to a nucleotide sequence of SEQ ID NOS:2, 4, 6, or 8 or the complement. In one embodiment, the nucleic acid consists of a portion of a nucleotide sequence of SEQ ID NOS:2, 4, 6, or 8 and the complement. The nucleic acid fragments of the invention are at least about 15, preferably at least about 18, 20, 23 or 25 nucleotides, and can be 30, 40, 50, 100, 200, 500 or more nucleotides in length. Longer fragments, for example, 30 or more nucleotides in length, which encode antigenic proteins or polypeptides described herein are useful.

In one embodiment, a nucleotide sequence of the present invention has at least 60%, 70%, 80%, 90%, 91%, 92%, 93%, 94%, 95%, 96%, 97%, 98%, 99% or greater identity to a nucleotide sequence shown in SEQ ID NOS:2, 4, 6, 8, 10, 12, 14, 16, 18, 20, 22, 24, or 26.

Furthermore, the invention provides polynucleotides that comprise a fragment of the full-length subtilase-like polynucleotides. The fragment can be single or double-stranded and can comprise DNA or RNA. The fragment can be derived from either the coding or the non-coding sequence.

In another embodiment an isolated nucleic acid encodes the entire coding region. In another embodiment the isolated nucleic acid encodes a sequence corresponding to the mature protein that may be from about amino acid 6 to the last amino acid. Other fragments include nucleotide sequences encoding the amino acid fragments described herein.

Thus, nucleic acid fragments further include sequences corresponding to the domains described herein, subregions also described, and specific functional sites. Nucleic acid fragments also include combinations of the domains, segments, and other functional sites described above. A person of ordinary skill in the art would be aware of the many permutations that are possible.

Where the location of the domains or sites have been predicted by computer analysis, one of ordinary skill would appreciate that the amino acid residues constituting these domains can vary depending on the criteria used to define the domains.

A fragment of the present invention comprises a nucleotide sequence consisting of nucleotides 1-100, 100-200, 200-300, 300-400, 400-500, 500-600, 600-700, 700-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3372 of SEQ ID NO:4.

In one embodiment, a fragment contains nucleotides 205-2277 of SEQ ID NO:4, set forth herein as SEQ ID NO:10.

Fragments include nucleotide sequences with substitutions at particular codons, thus encoding polypeptides with substitutions at particular amino acid residues. Techniques for the generation of site directed mutants are known in the art and set forth, for example, in Ausubel et al. (1998) Current Protocols in Molecular Biology, John Wiley & Sons.

In one embodiment, the codon CAT at position 673-675 of SEQ ID NO:10 is replaced by another codon. In another embodiment, the codon CAT at position 673-675 of SEQ ID NO:10 is replaced by the TGG codon. SEQ ID NO:12. The activity of the polypeptide encoded by this molecule is described below, in the Examples.

In one embodiment, the codon TCA at position 1153-1155 of SEQ ID NO:10 is replaced by another codon. In another embodiment, the codon TCA at position 1153-1155 of SEQ ID NO:10 is replaced by the codon GCA. The activity of the polypeptide encoded by this molecule is described below, in the Examples.

In one embodiment, both the codon CAT at position 673-675 of SEQ ID NO:10 and the codon TCA at position 1153-1155 of SEQ ID NO:10 are replaced by different codons. In another embodiment, both the codon CAT at position 673-675 of SEQ ID NO:10 and the codon TCA at position 1153-1155 of SEQ ID NO:10 are replaced by replaced by the TGG codon and the codon GCA, respectively. See SEQ ID NO:16. The activity of the polypeptide encoded by this molecule is described below, in the Examples.

Fragments can include truncations and deletion mutants. Techniques for the generation of such molecules are known in the art and set forth, for example, in Ausubel et al. (above).

In one embodiment, the nucleotide sequence set forth in SEQ ID NO:10 is truncated immediately after the ATG codon at position 1273-1275, set forth herein as SEQ ID NO:1. The activity of the polypeptide encoded by this molecule is described below, in the Examples. In another embodiment, the nucleotide sequence set forth in SEQ ID NO:10 is truncated immediately after the CAG codon at position 1357-1359 of SEQ iD NO:10, set forth herein as SEQ ID NO:20. The activity of the polypeptide encoded by this molecule is described below, in the Examples. In another embodiment, the nucleotide sequence set forth in SEQ ID NO:10 is truncated immediately after the GTC codon at position 1519-1521 of SEQ ID NO:10, set forth herein as SEQ ID NO:22. The activity of the polypeptide encoded by this molecule is described below, in the Examples.

In one embodiment, nucleotides 439 to 1275, inclusive, of SEQ ID NO:10 are deleted. This molecule is set forth herein as SEQ ID NO:24. The activity of the polypeptide encoded by this molecule is described below, in the Examples. In another embodiment, nucleotides 652 to 1176, inclusive, of SEQ ID NO:10 are deleted. This molecule is set forth herein as SEQ ID NO:26. The activity of the polypeptide encoded by this molecule is described below, in the Examples.

The invention also provides nucleic acid fragments that encode epitope bearing regions of the subtilase-like proteins described herein.

Nucleic acid fragments, according to the present invention, are not to be construed as encompassing those fragments that may have been disclosed prior to the invention.

Polynucleotide Uses

The nucleotide sequences of the present invention can be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The nucleic acid fragments of the invention provide probes or primers in assays such as those described below. “Probes” are oligonucleotides that hybridize in a base-specific manner to a complementary strand of nucleic acid. Such probes include polypeptide nucleic acids, as described in Nielsen et al. (1991) Science 254:1497-1500. Typically, a probe comprises a region of nucleotide sequence that hybridizes under highly stringent conditions to at least about 15, typically about 20-25, and more typically about 40, 50 or 75 consecutive nucleotides of a nucleic acid sequence shown in SEQ ID NOS:2, 4, 6, or 8 and the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

The polynucleotides are thus useful for probes, primers, and in biological assays.

Where the polynucleotides are used to assess subtilase-like protein properties or functions, such as in the assays described herein, all or less than all of the entire cDNA can be useful. Assays specifically directed to subtilase-like protein functions, such as assessing agonist or antagonist activity, encompass the use of known nucleotide fragments. Further, diagnostic methods for assessing subtilase-like protein function can also be practiced with any nucleotide fragment, including those fragments that may have been known prior to the invention. Similarly, in methods involving treatment of subtilase-like protein dysfunction, all nucleotide fragments are encompassed including those, which may have been known in the art.

The polynucleotides are useful as a hybridization probe for cDNA and genomic DNA to isolate a full-length cDNA and genomic clones encoding a polypeptide described in SEQ ID NOS:1, 3, 5, or 7 and to isolate cDNA and genomic clones that correspond to variants producing the same polypeptides shown in SEQ ID NOS:1, 3, 5, or 7 or the other variants described herein. Variants can be isolated from the same tissue and organism from which a polypeptide shown in SEQ ID NOS:1, 3, 5, or 7 were isolated, different tissues from the same organism, or from different organisms. This method is useful for isolating genes and cDNA that are developmentally-controlled and therefore may be expressed in the same tissue or different tissues at different points in the development of an organism.

The probe can correspond to any sequence along the entire length of the gene encoding the subtilase-like protein. Accordingly, it could be derived from 5′ noncoding regions, the coding region, and 3′ noncoding regions.

The nucleic acid probe can be, for example, a full-length cDNA of SEQ ID NOS:2, 4, 6, or 8, or a fragment thereof, such as an oligonucleotide of at least 12, 15, 30, 50, 100, 250 or 500 nucleotides in length and sufficient to specifically hybridize under stringent conditions to mRNA or DNA.

Fragments of the polynucleotides described herein are also useful to synthesize larger fragments or full-length polynucleotides described herein. For example, a fragment can be hybridized to any portion of an mRNA and a larger or full-length cDNA can be produced.

The fragments are also useful to synthesize antisense molecules of desired length and sequence.

Antisense nucleic acids of the invention can be designed using a nucleotide sequence of SEQ ID NOS:2, 4, 6, or 8, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Additionally, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

The nucleic acid molecules and fragments of the invention can also include other appended groups such as peptides (e.g., for targeting host cell subtilase-like proteins in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/0918) or the blood brain barrier (see, e.g., PCT Publication No. WO 89/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm Res. 5:539-549).

The polynucleotides are also useful as primers for PCR to amplify any given region of the polynucleotide of the invention.

The polynucleotides are also useful for constructing recombinant vectors. Such vectors include expression vectors that express a portion of, or all of, the subtilase-like polypeptides. Vectors also include insertion vectors, used to integrate into another polynucleotide sequence, such as into the cellular genome, to alter in situ expression of the genes and gene products. For example, an endogenous subtilase-like protein coding sequence can be replaced via homologous recombination with all or part of the coding region containing one or more specifically introduced mutations.

The polynucleotides are also useful for expressing antigenic portions of the subtilase-like proteins.

The polynucleotides are also useful as probes for determining the chromosomal positions of the polynucleotides by means of in situ hybridization methods, such as FISH. (For a review of this technique, see Verma et al. (1988) Human Chromosomes: A Manual of Basic Techniques (Pergamon Press, New York), and PCR mapping of somatic cell hybrids. The mapping of the sequences to chromosomes is an important first step in correlating these sequences with genes associated with disease.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library.) The relationship between a gene and a disease mapped to the same chromosomal region, can then be identified through linkage analysis (co-inheritance of physically adjacent genes), described in, for example, Egeland et al. ((1987) Nature 325:783-787).

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations, that are visible from chromosome spreads, or detectable using PCR based on that DNA sequence. Ultimately, complete sequencing of genes from several individuals can be performed to confirm the presence of a mutation and to distinguish mutations from polymorphisms.

The polynucleotide probes are also useful to determine patterns of the presence of the gene encoding the subtilase-like proteins and their variants with respect to tissue distribution, for example, whether gene duplication has occurred and whether the duplication occurs in all or only a subset of tissues. The genes can be naturally occurring or can have been introduced into a cell, tissue, or organism exogenously.

The polynucleotides are also useful for designing ribozymes corresponding to all, or a part, of the mRNA produced from genes encoding the polynucleotides described herein.

The polynucleotides are also useful for constructing host cells expressing a part, or all, of the subtilase-like polynucleotides and polypeptides.

The polynucleotides are also useful for constructing transgenic animals expressing all, or a part, of the subtilase-like polynucleotides and polypeptides.

The polynucleotides are also useful for making vectors that express part, or all, of the subtilase-like polypeptides.

The polynucleotides are also useful as hybridization probes for determining the level of nucleic acid expression. Accordingly, the probes can be used to detect the presence of, or to determine levels of, subtilase-like nucleic acid in cells, tissues, and in organisms. The nucleic acid whose level is determined can be DNA or RNA. Accordingly, probes corresponding to the polypeptides described herein can be used to assess gene copy number in a given cell, tissue, or organism. This is particularly relevant in cases in which there has been an amplification of the gene of the invention.

Alternatively, the probe can be used in an in situ hybridization context to assess the position of extra copies of the gene, as on extrachromosomal elements or as integrated into chromosomes in which the gene is not normally found, for example as a homogeneously staining region.

These uses are relevant for diagnosis of disorders involving an increase or decrease in subtilase-like protein expression relative to normal, such as a proliferative disorder, a differentiative or developmental disorder, or a hematopoietic disorder.

As such, the gene is particularly relevant for the treatment of disorders including but not limited to those disclosed herein.

Disorders in which subtilase-like protein expression is particularly relevant also include, but are not limited to, disorders involving programmed cell death, such as those disclosed herein, disorders involving obesity, liver disorders, and disorders associated with mitochondrial dysfunction as a result of defects in proprotein processing.

Thus, the present invention provides a method for identifying a disease or disorder associated with aberrant expression or activity of the nucleic acid, in which a test sample is obtained from a subject and nucleic acid (e.g., mRNA, genomic DNA) is detected, wherein the presence of the nucleic acid is diagnostic for a subject having or at risk of developing a disease or disorder associated with aberrant expression or activity of the nucleic acid.

One aspect of the invention relates to diagnostic assays for determining nucleic acid expression as well as activity in the context of a biological sample (e.g., blood, serum, cells, tissue) to determine whether an individual has a disease or disorder, or is at risk of developing a disease or disorder, associated with aberrant nucleic acid expression or activity. Such assays can be used for prognostic or predictive purpose to thereby prophylactically treat an individual prior to the onset of a disorder characterized by or associated with expression or activity of the nucleic acid molecules.

In vitro techniques for detection of mRNA include Northern hybridizations and in situ hybridizations. In vitro techniques for detecting DNA includes Southern hybridizations and in situ hybridization.

Probes can be used as a part of a diagnostic test kit for identifying cells or tissues that express the subtilase-like protein, such as by measuring the level of a subtilase-like protein-encoding nucleic acid in a sample of cells from a subject e.g., mRNA or genomic DNA, or determining if the gene encoding the protein has been mutated.

Nucleic acid expression assays are useful for drug screening to identify compounds that modulate expression of the nucleic acid of the invention (e.g., antisense, polypeptides, peptidomimetics, small molecules or other drugs). A cell is contacted with a candidate compound and the expression of mRNA determined. The level of expression of the mRNA in the presence of the candidate compound is compared to the level of expression of the mRNA in the absence of the candidate compound. The candidate compound can then be identified as a modulator of nucleic acid expression based on this comparison and be used, for example to treat a disorder characterized by aberrant nucleic acid expression. The modulator can bind to the nucleic acid or indirectly modulate expression, such as by interacting with other cellular components that affect nucleic acid expression.

Modulatory methods can be performed in vitro (e.g., by culturing the cell with the agent) or, alternatively, in vivo (e.g., by administering the gent to a subject) in patients or in transgenic animals.

The invention thus provides a method for identifying a compound that can be used to treat a disorder associated with nucleic acid expression of the gene for the subtilase-like protein. The method typically includes assaying the ability of the compound to modulate the expression of the nucleic acid and thus identifying a compound that can be used to treat a disorder characterized by undesired expression of the nucleic acid.

The assays can be performed in cell-based and cell-free systems. Cell-based assays include cells naturally expressing the nucleic acid or recombinant cells genetically engineered to express specific nucleic acid sequences.

Alternatively, candidate compounds can be assayed in vivo in patients or in transgenic animals.

The assay for nucleic acid expression can involve direct assay of nucleic acid levels, such as mRNA levels, or on collateral compounds such as free propeptide, mature substrate, and any of the downstream components or cellular events that result from subtilase-like protein expression, including but not limited to those disclosed hereinabove. Further, the expression of genes that are up- or down-regulated in response to the subtilase-like protein expression can also be assayed. In this embodiment the regulatory regions of these genes can be operably linked to a reporter gene such as luciferase.

Thus, modulators of subtilase-like gene expression can be identified in a method wherein a cell is contacted with a candidate compound and the expression of mRNA determined. The level of expression of the mRNA in the presence of the candidate compound is compared to the level of expression of the mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Accordingly, the invention provides methods of treatment, with the nucleic acid as a target, using a compound identified through drug screening as a gene modulator to modulate the nucleic acid expression. Modulation includes both up-regulation (i.e. activation or agonization) or down-regulation (suppression or antagonization) or effects on nucleic acid activity (e.g. when nucleic acid is mutated or improperly modified). Treatment is of disorders characterized by aberrant expression or activity of the nucleic acid.

The gene is particularly relevant for the treatment of disorders involving obesity, liver function, mitochondrial dysfunction, and programmed cell death, and in particular, neuronal cell death, especially in brain.

Alternatively, a modulator for the nucleic acid expression can be a small molecule or drug identified using the screening assays described herein as long as the drug or small molecule inhibits the nucleic acid expression.

The polynucleotides are also useful for monitoring the effectiveness of modulating compounds on the expression or activity of the gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Monitoring can be, for example, as follows: (i) obtaining a pre-administration sample from a subject prior to administration of the agent; (ii) detecting the level of expression of a specified mRNA or genomic DNA of the invention in the pre-administration sample; (iii) obtaining one or more post-administration samples from the subject; (iv) detecting the level of expression or activity of the mRNA or genomic DNA in the post-administration samples; (v) comparing the level of expression or activity of the mRNA or genomic DNA in the pre-administration sample with the mRNA or genomic DNA in the post-administration sample or samples; and (vi) increasing or decreasing the administration of the agent to the subject accordingly.

The polynucleotides are also useful in diagnostic assays for qualitative changes in the nucleic acid, and particularly in qualitative changes that lead to pathology. The polynucleotides can be used to detect mutations in the genes of the invention and gene expression products such as mRNA. The polynucleotides can be used as hybridization probes to detect naturally-occurring genetic mutations in the gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the gene associated with a dysfunction provides a diagnostic tool for an active disease or susceptibility to disease when the disease results from overexpression, underexpression, or altered expression of a subtilase-like protein.

Mutations in the gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

Alternatively, mutations in the gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and S1 protection or the chemical cleavage method.

Furthermore, sequence differences between a mutant gene and a wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the gene include methods in which protection from cleavage agents is used to detect mismatched bases in RNA/RNA or RNA/DNA duplexes (Myers et al. (1985) Science 230:1242); Cotton et al. (1988) PNAS 85:4397; Saleeba et al. (1992) Meth. Enzymol. 217:286-295), electrophoretic mobility of mutant and wild type nucleic acid is compared (Orita et al. (1989) PNAS 86:2766; Cotton et al. (1993) Mutat. Res. 285:125-144; and Hayashi et al. (1992) Genet. Anal. Tech. Appl. 9:73-79), and movement of mutant or wild-type fragments in polyacrylamide gels containing a gradient of denaturant is assayed using denaturing gradient gel electrophoresis (Myers et al. (1985) Nature 313:495). The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5). Examples of other techniques for detecting point mutations include, selective oligonucleotide hybridization, selective amplification, and selective primer extension.

In other embodiments, genetic mutations can be identified by hybridizing a sample and control nucleic acids, e.g., DNA or RNA, to high density arrays containing hundreds or thousands of oligonucleotide probes (Cronin et al. (1996) Human Mutation 7:244-255; Kozal et al. (1996) Nature Medicine 2:753-759). For example, genetic mutations can be identified in two dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

The polynucleotides are also useful for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the gene that results in altered affinity for substrate or propeptide could result in an excessive or decreased drug effect with standard concentrations of these components that activates/inhibits the subtilase-like protein. Accordingly, the polynucleotides described herein can be used to assess the mutation content of the gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The methods can involve obtaining a control biological sample from a control subject, contacting the control sample with a compound or agent capable of detecting mRNA, or genomic DNA, such that the presence of mRNA or genomic DNA is detected in the biological sample, and comparing the presence of mRNA or genomic DNA in the control sample with the presence of mRNA or genomic DNA in the test sample.

The polynucleotides are also useful for chromosome identification when the sequence is identified with an individual chromosome and to a particular location on the chromosome. First, the DNA sequence is matched to the chromosome by in situ or other chromosome-specific hybridization. Sequences can also be correlated to specific chromosomes by preparing PCR primers that can be used for PCR screening of somatic cell hybrids containing individual chromosomes from the desired species. Only hybrids containing the chromosome containing the gene homologous to the primer will yield an amplified fragment. Sublocalization can be achieved using chromosomal fragments. Other strategies include prescreening with labeled flow-sorted chromosomes and preselection by hybridization to chromosome-specific libraries. Further mapping strategies include fluorescence in situ hybridization, which allows hybridization with probes shorter than those traditionally used. Reagents for chromosome mapping can be used individually to mark a single chromosome or a single site on the chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

The polynucleotides can also be used to identify individuals from small biological samples. This can be done for example using restriction fragment-length polymorphism (RFLP) to identify an individual. Thus, the polynucleotides described herein are useful as DNA markers for RFLP (See U.S. Pat. No. 5,272,057).

Furthermore, the subtilase-like protein sequence can be used to provide an alternative technique, which determines the actual DNA sequence of selected fragments in the genome of an individual. Thus, the subtilase-like protein sequences described herein can be used to prepare two PCR primers from the 5′ and 3′ ends of the sequences. These primers can then be used to amplify DNA from an individual for subsequent sequencing.

Panels of corresponding DNA sequences from individuals prepared in this manner can provide unique individual identifications, as each individual will have a unique set of such DNA sequences. It is estimated that allelic variation in humans occurs with a frequency of about once per each 500 bases. Allelic variation occurs to some degree in the coding regions of these sequences, and to a greater degree in the noncoding regions. The subtilase-like protein sequences can be used to obtain such identification sequences from individuals and from tissue. The sequences represent unique fragments of the human genome. Each of the sequences described herein can, to some degree, be used as a standard against which DNA from an individual can be compared for identification purposes.

If a panel of reagents from the sequences is used to generate a unique identification database for an individual, those same reagents can later be used to identify tissue from that individual. Using the unique identification database, positive identification of the individual, living or dead, can be made from extremely small tissue samples.

The polynucleotides can also be used in forensic identification procedures. PCR technology can be used to amplify DNA sequences taken from very small biological samples, such as a single hair follicle, body fluids (e.g. blood, saliva, or semen). The amplified sequence can then be compared to a standard allowing identification of the origin of the sample.

The polynucleotides can thus be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region are particularly useful since greater polymorphism occurs in the noncoding regions, making it easier to differentiate individuals using this technique.

The polynucleotides can further be used to provide polynucleotide reagents, e.g., labeled or labelable probes which can be used in, for example, an in situ hybridization technique, to identify a specific tissue. This is useful in cases in which a forensic pathologist is presented with a tissue of unknown origin. Panels of subtilase-like protein probes can be used to identify tissue by species and/or by organ type.

In a similar fashion, these primers and probes can be used to screen tissue culture for contamination (i.e. screen for the presence of a mixture of different types of cells in a culture).

Alternatively, the polynucleotides can be used directly to block transcription or translation of subtilase-like gene sequences by means of antisense or ribozyme constructs. Thus, in a disorder characterized by abnormally high or undesirable subtilase-like gene expression, nucleic acids can be directly used for treatment.

The polynucleotides are thus useful as antisense constructs to control expression of the gene in cells, tissues, and organisms. A DNA antisense polynucleotide is designed to be complementary to a region of the gene involved in transcription, preventing transcription and hence production of subtilase-like protein. An antisense RNA or DNA polynucleotide would hybridize to the mRNA and thus block translation of mRNA into subtilase-like protein.

Examples of antisense molecules useful to inhibit nucleic acid expression include antisense molecules complementary to a fragment of the 5′ untranslated region of SEQ ID NOS:2, 4, 6, or 8 which also includes the start codon and antisense molecules which are complementary to a fragment of the 3′ untranslated region of SEQ ID NOS:2, 4, 6, or 8.

Alternatively, a class of antisense molecules can be used to inactivate mRNA in order to decrease expression of subtilase-like nucleic acid. Accordingly, these molecules can treat a disorder characterized by abnormal or undesired expression of the nucleic acid of the invention. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the subtilase-like protein.

The polynucleotides also provide vectors for gene therapy in patients containing cells that are aberrant in gene expression. Thus, recombinant cells, which include the patient's cells that have been engineered ex vivo and returned to the patient, are introduced into an individual where the cells produce the desired subtilase-like protein to treat the individual.

The invention also encompasses kits for detecting the presence of the nucleic acid in a biological sample. For example, the kit can comprise reagents such as a labeled or labelable nucleic acid or agent capable of detecting the nucleic acid in a biological sample; means for determining the amount of the nucleic acid in the sample; and means for comparing the amount of the nucleic acid in the sample with a standard. The compound or agent can be packaged in a suitable container. The kit can further comprise instructions for using the kit to detect subtilase-like mRNA or DNA.

Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exists in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

Vectors/Host Cells

The invention also provides vectors containing the polynucleotides of the invention. The term “vector” refers to a vehicle, preferably a nucleic acid molecule that can transport the polynucleotides. When the vector is a nucleic acid molecule, the polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the polynucleotides of the invention. Alternatively, the vector may integrate into the host cell genome and produce additional copies of the polynucleotides when the host cell replicates.

The invention provides vectors for the maintenance (cloning vectors) or vectors for expression (expression vectors) of the polynucleotides of the invention. The vectors can function in procaryotic or eukaryotic cells or in both (shuttle vectors).

Expression vectors contain cis-acting regulatory regions that are operably linked in the vector to the polynucleotides of the invention such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the polynucleotides from the vector. Alternatively, a transacting factor may be supplied by the host cell. Finally, a trans-acting factor can be produced from the vector itself.

It is understood, however, that in some embodiments, transcription and/or translation of the polynucleotides of the invention can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd. ed., Cold Spring Harbor Laboratory Press, Cold Spring Harbor, N.Y.).

A variety of expression vectors can be used to express a polynucleotide of the invention. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxyiruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al. (1989) Molecular Cloning: A Laboratory Manual 2nd. ed., Cold Spring Harbor Laboratory Press, Cold Spring Harbor, N.Y.

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The polynucleotides of the invention can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the subtilase-like polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al. (1988) Gene 69:301-315) and pET 1 id (Studier et al. (1990) Gene Expression Technology: Methods in Enzymology 185:60-89).

Recombinant protein expression can be maximized in a host bacteria by providing a genetic background wherein the host cell has an impaired capacity to proteolytically cleave the recombinant protein. (Gottesman, S. (1990) Gene Expression Technology: Methods in Enzymology 185, Academic Press, San Diego, Calif. 119-128). Alternatively, the sequence of the polynucleotide of interest can be altered to provide preferential codon usage for a specific host cell, for example E. coli. (Wada et al. (1992) Nucleic Acids Res. 20:2111-2118).

The polynucleotides of the invention can also be expressed by expression vectors that are operative in yeast. Examples of vectors for expression in yeast e.g., S. cerevisiae include pYepSecl (Baldari et al. (1987) EMBO J. 6:229-234), pMFa (Kurjan et al. (1982) Cell 30:933-943), pJRY88 (Schultz et al. (1987) Gene 54:113-123), and pYES2 (Invitrogen Corporation, San Diego, Calif.).

The polynucleotides can also be expressed in insect cells using, for example, baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow et al. (1989) Virology 170:31-39).

In certain embodiments of the invention, the polynucleotides described herein are expressed in mammalian cells using mammalian expression vectors. Examples of mammalian expression vectors include pCDM8 (Seed, B. (1987) Nature 329:840) and pMT2PC (Kaufman et al. (1987) EMBO J. 6:187-195).

The expression vectors listed herein are provided by way of example only of the well-known vectors available to those of ordinary skill in the art that would be useful to express the polynucleotides of the invention. The person of ordinary skill in the art would be aware of other vectors suitable for maintenance propagation or expression of the polynucleotides described herein. These are found for example in Sambrook et al. (1989) Molecular Cloning: A Laboratory Manual 2nd, ed., Cold Spring Harbor Laboratory, Cold Spring Harbor Laboratory Press, Cold Spring Harbor, N.Y.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook et al. (Molecular Cloning: A Laboratory Manual, 2d ed., Cold Spring Harbor Laboratory, Cold Spring Harbor Laboratory Press, Cold Spring Harbor, N.Y.).

Host cells can contain more than one vector. Thus, different nucleotide sequences can be introduced on different vectors of the same cell. Similarly, the polynucleotides of the invention can be introduced either alone or with other polynucleotides that are not related to the polynucleotides of the invention such as those providing trans-acting factors for expression vectors. When more than one vector is introduced into a cell, the vectors can be introduced independently, co-introduced or joined to the subtilase-like polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the subtilase-like polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Uses of Vectors and Host Cells

It is understood that “host cells” and “recombinant host cells” refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

The host cells expressing the polypeptides described herein, and particularly recombinant host cells, have a variety of uses. First, the cells are useful for producing subtilase-like proteins or polypeptides that can be further purified to produce desired amounts of subtilase-like protein or fragments. Thus, host cells containing expression vectors are useful for polypeptide production.

Host cells are also useful for conducting cell-based assays involving the subtilase-like protein or subtilase-like protein fragments. Thus, a recombinant host cell expressing a native subtilase-like protein is useful to assay for compounds that stimulate or inhibit the subtilase-like protein function. This includes substrate binding, gene expression at the level of transcription or translation, propeptide interaction, and downstream components of pathways affected by subtilase-like protein activation.

Host cells are also useful for identifying subtilase-like protein mutants in which these functions are affected. If the mutants naturally occur and give rise to a pathology, host cells containing the mutations are useful to assay compounds that have a desired effect on the mutant subtilase-like protein (for example, stimulating or inhibiting function) which may not be indicated by their effect on the native subtilase-like protein.

Recombinant host cells are also useful for expressing the chimeric polypeptides described herein to assess compounds that activate or suppress activation by means of a heterologous domain, segment, site, and the like, as disclosed herein.

Further, mutant subtilase-like proteins can be designed in which one or more of the various functions is engineered to be increased or decreased and used to augment or replace subtilase-like proteins in an individual. Thus, host cells can provide a therapeutic benefit by replacing an aberrant subtilase-like protein or providing an aberrant subtilase-like protein that provides a therapeutic result. In one embodiment, the cells provide a subtilase-like protein that is abnormally active.

In another embodiment, the cells provide subtilase-like proteins that are abnormally inactive. These can compete with the endogenous subtilase-like proteins in the individual.

In another embodiment, cells expressing a subtilase-like protein that cannot be activated are introduced into an individual in order to compete with the endogenous one.

Homologously recombinant host cells can also be produced that allow the in situ alteration of the endogenous polynucleotide sequence in a host cell genome. The host cell includes, but is not limited to, a stable cell line, cell in vivo, or cloned microorganism. This technology is more fully described in WO 93/09222, WO 91/12650, WO 91/06667, U.S. Pat. No. 5,272,071, and U.S. Pat. No. 5,641,670. Briefly, specific polynucleotide sequences corresponding to the polynucleotides of the invention or sequences proximal or distal to a gene of the invention are allowed to integrate into a host cell genome by homologous recombination where expression of the gene can be affected. In one embodiment, regulatory sequences are introduced that either increase or decrease expression of an endogenous sequence. Accordingly, a subtilase-like protein can be produced in a cell not normally producing it. Alternatively, increased expression of subtilase-like protein can be effected in a cell normally producing the protein at a specific level. Further, expression can be decreased or eliminated by introducing a specific regulatory sequence. The regulatory sequence can be heterologous to the subtilase-like protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant subtilase-like proteins. Such mutations could be introduced, for example, into the specific functional regions such as the ligand-binding site.

In one embodiment, the host cell can be a fertilized oocyte or embryonic stem cell that can be used to produce a transgenic animal containing the altered gene. Alternatively, the host cell can be a stem cell or other early tissue precursor that gives rise to a specific subset of cells and can be used to produce transgenic tissues in an animal. See also Thomas et al. (1987) Cell 51:503 or a description of homologous recombination vectors. The vector is introduced into an embryonic stem cell line (e.g., by electroporation) and cells in which the introduced gene has homologously recombined with the endogenous subtilase-like gene is selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a subtilase-like protein and identifying and evaluating modulators of subtilase-like protein activity.

Other examples of transgenic animals include non-human primates, sheep, dogs, cows, goats, chickens, and amphibians.

In one embodiment, a host cell is a fertilized oocyte or an embryonic stem cell into which a polynucleotide sequence of the invention has been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the nucleotide sequences of the invention can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the subtilase-like protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems, which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of S. cerevisiae (O'Gorman et al. (1991) Science 251:1351-1355). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could affect substrate binding, subtilase-like protein activation, and translocation, may not be evident from in vitro cell-free or cell-based assays. Accordingly, it is useful to provide non-human transgenic animals to assay in vivo subtilase-like protein function, including substrate interaction, the effect of specific mutant subtilase-like proteins on subtilase-like protein function and substrate interaction, and the effect of chimeric subtilase-like proteins. It is also possible to assess the effect of null mutations, that is mutations that substantially or completely eliminate one or more subtilase-like protein functions.

In general, methods for producing transgenic animals include introducing a nucleic acid sequence according to the present invention, the nucleic acid sequence capable of expressing the subtilase-like protein in a transgenic animal, into a cell in culture or in vivo. When introduced in vivo, the nucleic acid is introduced into an intact organism such that one or more cell types and, accordingly, one or more tissue types, express the nucleic acid encoding the subtilase-like protein. Alternatively, the nucleic acid can be introduced into virtually all cells in an organism by transfecting a cell in culture, such as an embryonic stem cell, as described herein for the production of transgenic animals, and this cell can be used to produce an entire transgenic organism. As described, in a further embodiment, the host cell can be a fertilized oocyte. Such cells are then allowed to develop in a female foster animal to produce the transgenic organism.

Pharmaceutical Compositions

The subtilase-like protein, modulators of the protein, nucleic acid molecules and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

The term “administer” is used in its broadest sense and includes any method of introducing the compositions of the present invention into a subject. This includes producing polypeptides or polynucleotides in vivo as by transcription or translation, in vivo, of polynucleotides that have been exogenously introduced into a subject. Thus, polypeptides or nucleic acids produced in the subject from the exogenous compositions are encompassed in the term “administer.”

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a subtilase-like protein or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

This invention is further illustrated by the following examples, which should not be construed as limiting. The contents of all references, patents and published patent applications cited throughout this application are incorporated herein by reference.

### EXAMPLES

### Example 1

### Transcriptional Characterization of the NARC1 Molecule

In neurons, programmed cell death is an essential component of neuronal development (Jacobson et al. 1997; Pettmann and Henderson (1998)) and has been associated with many forms of neurodegeneration (Hetts, (1998)). In the cerebellum, granule cell development occurs postnatally. The final number of neurons represents the combined effects of additive processes such as cell division and subtractive processes such as target-related programmed cell death. Depolarization due to high concentrations (25 mM) of extracellular potassium (K⁺) promotes the survival of cerebellar granule neurons (CGNs) in vitro. CGNs maintained in serum containing medium with high K⁺ will undergo programmed cell death when switched to serum-free medium with low K⁺ (5 mM) (D'Mello et al. 1993; Miller and Johnson Jr. 1996). The resulting programmed cell death has a transcriptional component that can be blocked by inhibitors of new RNA synthesis (Galli et al. 1995; Schulz et al. 1996).

As previously disclosed in U.S. Provisional Patent application No. 60/161,188, the inventors in that application constructed a brain-biased and programmed cell death-enriched clone set by arraying ˜7300 consolidated ESTs from two cDNA libraries cloned from rat frontal cortex and differentiated PC12 cells deprived of nerve growth factor (NGF), and >300 genes that are known markers for the central nervous system and/or programmed cell death. They reproducibly and simultaneously monitored the expression of the genes at 1, 3, 6, 12, and 24 hours after K⁺ withdrawal. They then categorized the regulated genes by time course expression pattern to identify cellular processes mobilized by CGN programmed cell death at the RNA level. In particular they focused on the expression profiles of many known pro- and anti-apoptotic regulatory proteins, including transcription factors, Bcl-2 family members, caspases, cyclins, heat shock proteins (HSPs), inhibitors of apoptosis (IAPs), growth factors and receptors, other signal transduction molecules, p53, superoxide dismutases (SODs), and other stress response genes. Finally, they compared the time courses of regulated genes induced by K⁺ withdrawal in the presence or absence of serum to those induced by glutamate toxicity. Thus, they identified a restricted set of relevant genes regulated by multiple models of programmed cell death in CGNs. These genes included the rat NARC1.

Using the brain-biased, programmed cell death nucleic acid-enriched Smart Chip, global mRNA expression was profiled throughout a time course of KCl/serum-withdrawal-induced cell death in primary cultures of CGNs. The transcription-dependent CGN programmed cell death was coordinated, resulting in less than 30% survival at 24 hours post-withdrawal as quantified by cell counting (data not shown). RNA samples, designated “treated”, were isolated at 1, 3, 6, 12, and 24 hours after switching post-natal day eight CGNs from medium containing 5% serum and 25 mM KCl to serum-free medium with 5 mM KCl. For controls, the 5% serum/25 mM KCl medium was replaced, and “sham” RNA at 1, 3, 6, 12, and 24 hours was isolated.

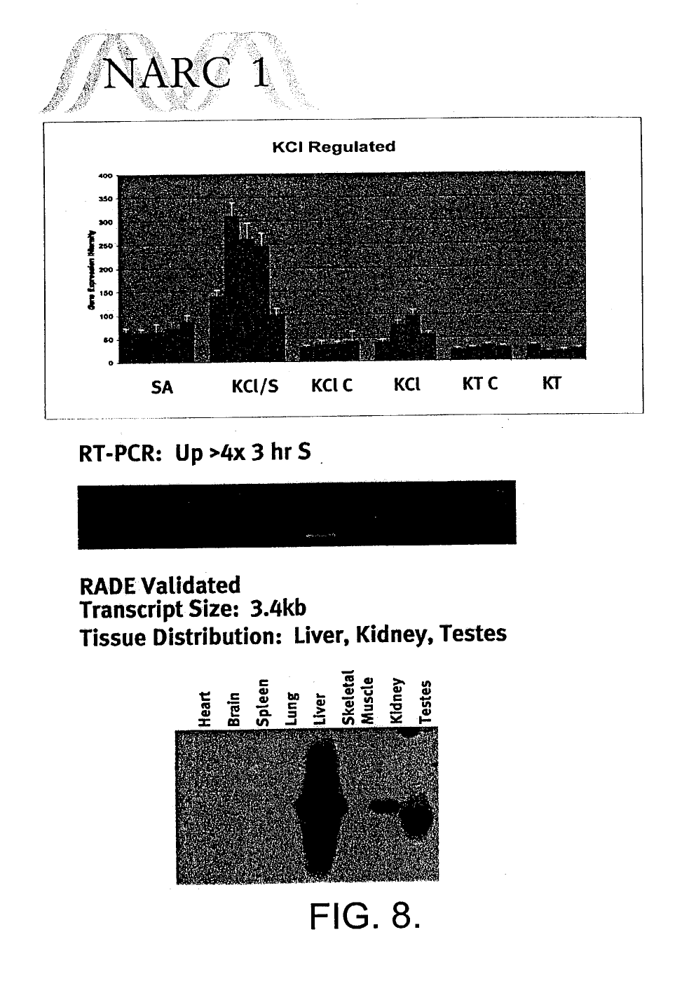

A third model of programmed cell death used to assay NARC expression involves kainate treatment. See, for example, FIG. 8 herein, and U.S. Provisional Application No. 60/161,188, incorporated herein by reference for teaching (among other things) this model.

FIG. 8 shows the result of experiments designed to characterize transcriptional characteristics for the rat NARC1 gene. For the top panel, the results show that, for NARC1, gene expression peaked at 3 hr both in the potassium/serum withdrawal paradigm and also in the potassium alone withdrawal paradigm. The conclusion from these results is that NARC1 is a gene that is regulated by the transcriptionally-dependent models of programmed cell death in the cerebellar granular neurons.

RT-PCR performed with sequence-specific primers for NARC1, confirm, for one of the time points in the top panel, the results in the top panel. That is, these RT-PCR results show that at 3 hr after potassium/serum withdrawal, the same regulation was observed using an independent technique. Accordingly, RT-PCR results confirm an upregulation of greater than four-fold for the NARC1 gene 3 hours after withdrawal.

The transcript size is 3.4 kb, which is the length of the sequence determined for NARC1.

The tissue distribution shows high expression in the liver with lower levels of expression in the testes and in the kidney. There is little expression in brain. This fits the disease model which is that this gene is expressed only when neurons are undergoing cellular distress and cell death. Accordingly, the gene provides a drug target for apoptosis/programmed cell death.

Confirmation of NARC1 Transcriptional Regulation by Taqman™ Analysis.

Transcriptional regulation of NARC1 was analyzed by a time course study of KCl/serum-withdrawal-induced cell death in primary cultures of CGNs. In particular, primary cultures of CGNs were incubated in media containing 5 mM KCl with no serum over a 24-hour period. Control cultures of CGNs were cultured over the same period in media containing 25 mM KCl with serum.

Cell samples were taken at 1, 3, 6, 12, and 24 hours, and RNA was isolated therefrom. Expression of NARC1, as well as Hsp70 and Caspase 3, was then studied by Taqman™ analysis. Hsp70 and Caspase 3 were included as controls: both genes are known to be transcriptionally regulated during programmed cell-death. Taqman™ analysis was carried out according to the manufacturer's protocols.

As expected, the Caspase 3 and Hsp 70 controls showed elevated levels of expression during the course of the experiment. NARC1 also demonstrated increased expression levels, peaking at 6 hours, thereby confirming that NARC1 is transcriptionally regulated during programmed cell death.

### Example 2

### NARC1 Induces Programmed Cell Death When Transiently Expressed in CGNs

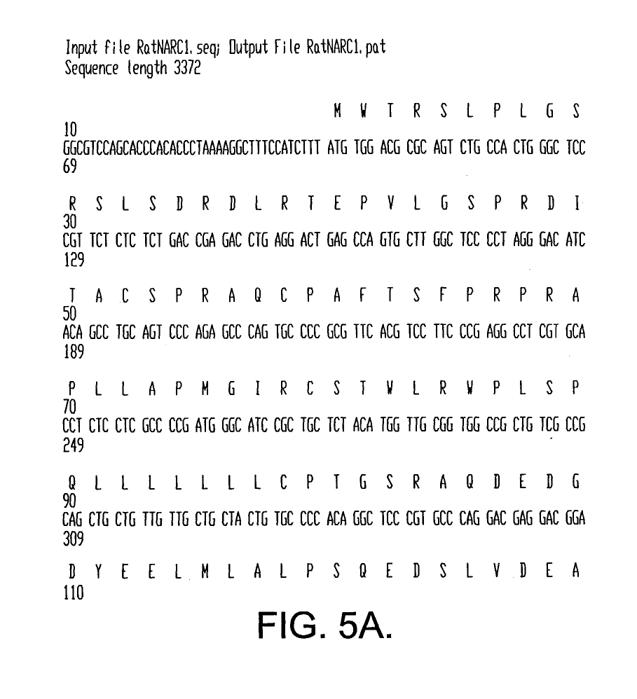

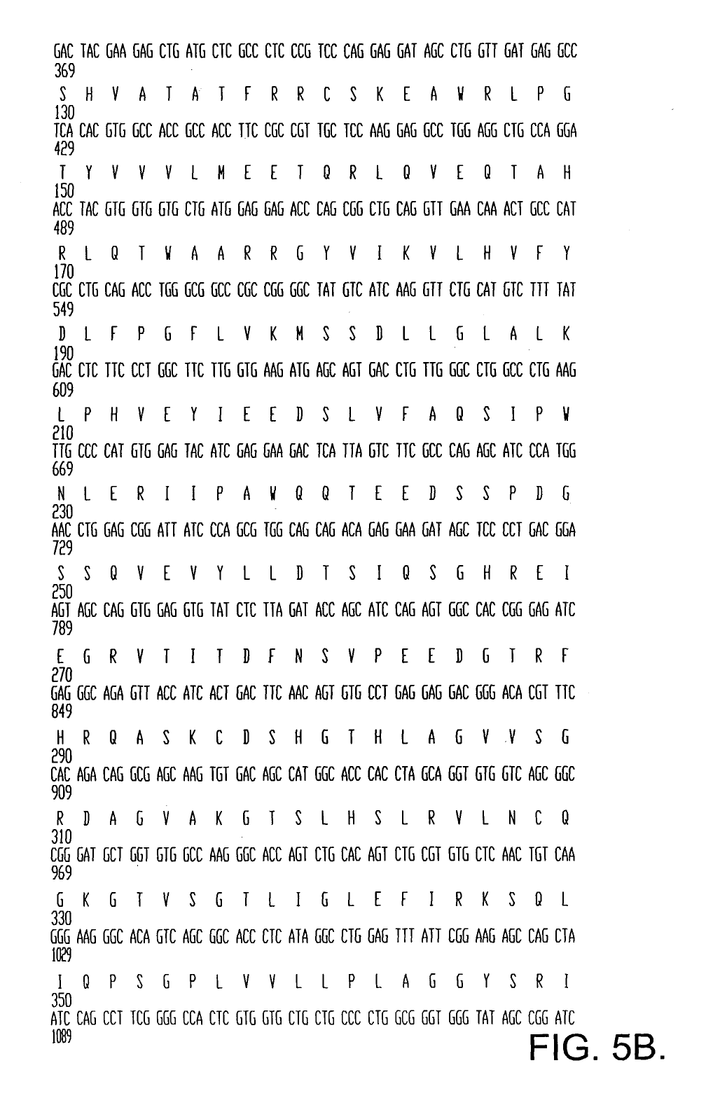

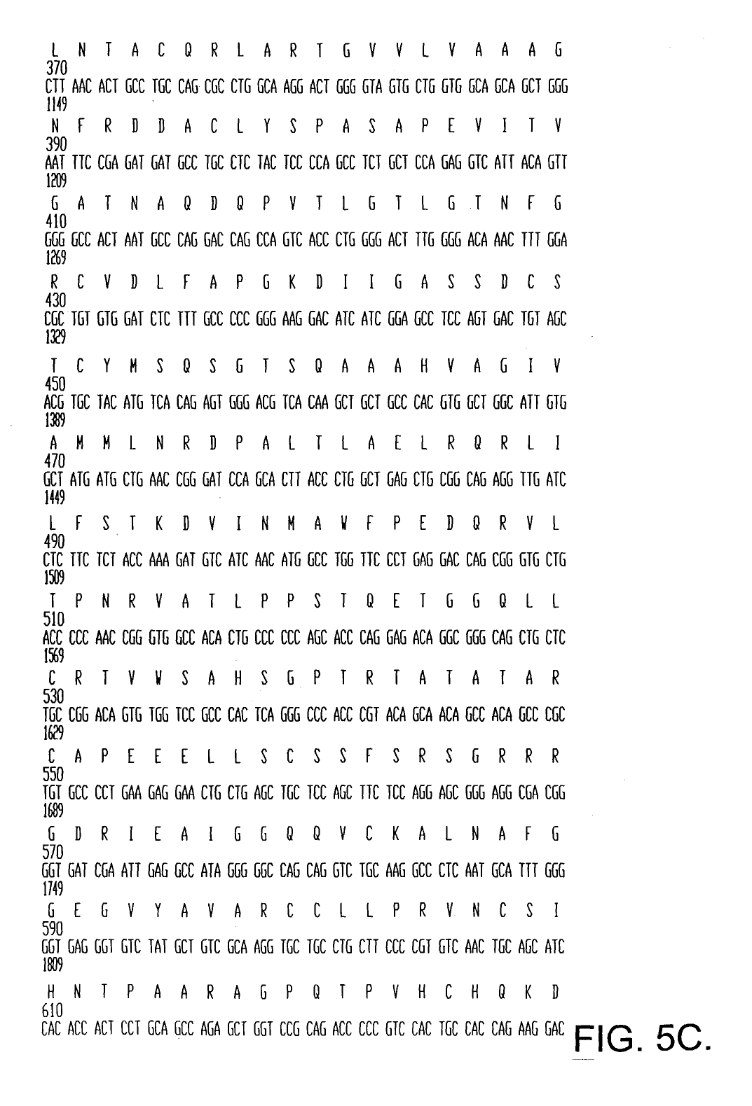

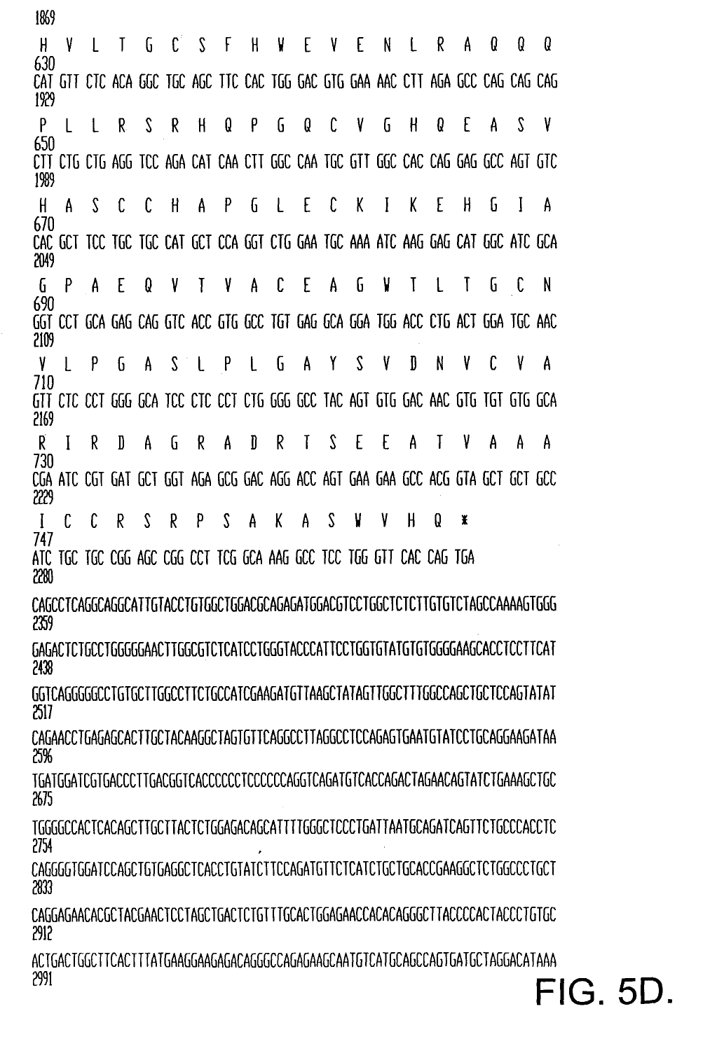

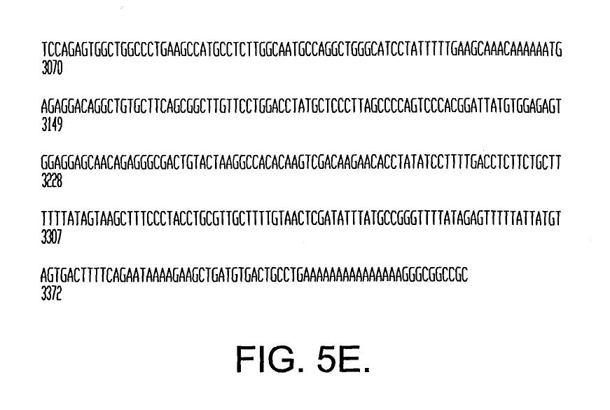

Cerebellar granule neurons (CGNs) were transfected with a NARC1-enhanced green fluorescent protein (EGFP) expression construct (with the EGFP sequences located at the carboxy terminus of the fusion protein). Unless otherwise indicated, the construct utilized in the following experiments contained 2073 base pairs of the rat NARC1 transcript set forth in FIG. 5. Specifically, this corresponds to nucleotides 205-2277 and amino acid residues 56-746 of SEQ ID NO:4 and SEQ ID NO:3, respectively. The polypeptide sequence encoded by the NARC1 insert utilized herein has been set forth as SEQ ID NO:9 herein. The nucleotide sequence of this insert has been set forth as SEQ ID NO:10.

Following transient expression of this construct in CGNs, the percentage of GFP positive and GFP negative cells undergoing apoptosis was determined by Laser-Scanning Cytometry. NARC1-EGFP cells showed an increase in apoptosis, demonstrating the molecules pro-apoptotic activity. In addition, the pro-apoptotic effect of the NARC1-EGFP construct was partially rescued by the administration of 100 μM boc-aspartyl-fluoromethylketone (BAF), a poly-caspase inhibitor. Accordingly, the NARC1 molecule was subjected to in-depth functional analysis, as set forth in detail below.

### Example 3

### Functional Analysis of the NARC1 Molecule

Generation of NARC1 Mutants.

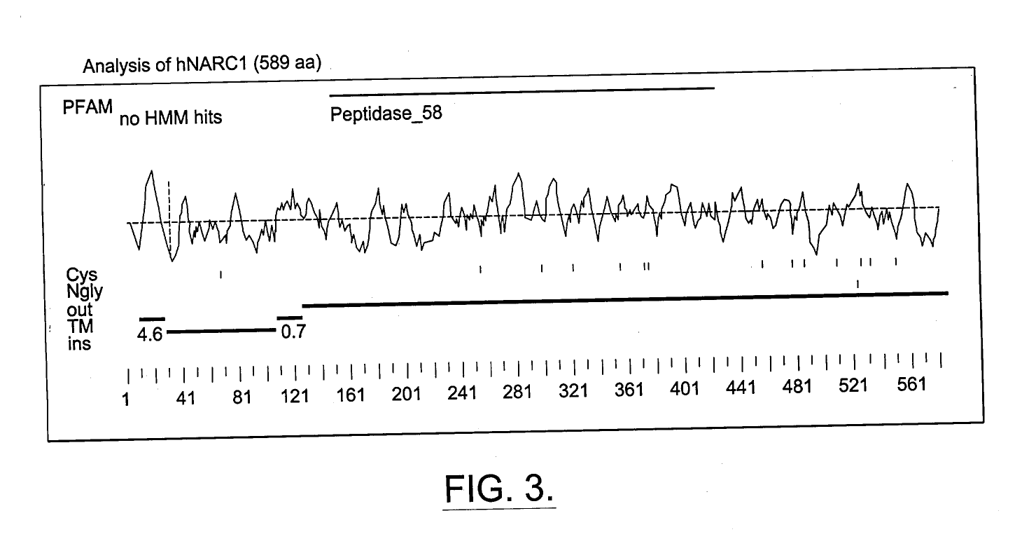

As indicated above, the NARC1 molecule was assigned to the subtilase-like family of molecules by bioinformatic analysis. See page 16, above; see also FIG. 3, setting forth the PFAM peptidase_S8 (subtilase) domain. (For a description of the Pfam database of multiple sequence alignments and HMMs, and its use in large-scale genome analysis, see Sonnhammer et al. (1997) Proteins 28:405-420.) The identity of the NARC1 functional domains was predicted based upon this assignment and the alignments described above. These assignments are summarized in Table 2, below.

TABLE 2
Identity of the NARC1 Protein Domains
Based on Bioinformatics and Polypeptide Sequencing*

| | | |
|---|---|---|
| 1) | Signal Peptide | M1-A30 |
| 2) | Pro-domain | Q31-Q151† |
| 3) | Subtilase Domain | S152†-M425 |
| 4) | Cysteine-Rich Region (CRR) | A426-Q691 |

*Residue number corresponds to amino acid residues of SEQ ID NO:9.
†The bioinformatics assigned the pro-domain as Q31-S146.

A set of site-directed mutants, as well as a series of deletion and truncation mutants, were then generated based upon the bioinformatics considerations. Techniques and protocols for the generation of site-directed mutants, deletion mutants, and truncation mutants, are known in the art, non-limiting examples of which can be found in, e.g., Ausubel et al. (1998) Current Protocols in Molecular Biology (John Wiley & Sons). The various mutants of NARC1 are summarized in Table 3 below.

TABLE 3
Identity of Site-Directed NARC1 Mutants

| | | | | SEQ ID NO: |
|---|---|---|---|---|
| Amino Acid | | Resulting | Effect of | (Polypeptide/ |
| Residue Affected* | | Structure | Alteration | Nucleotide) |
| 1 | H225W | Full-length | Mutation of the | SEQ ID NO:11/ |
| | | point mutant. | active site | SEQ ID NO:12 |
| | | | histidine. | |
| 2 | S385A | Full-length | Mutation of the | SEQ ID NO:13/ |
| | | point mutant. | active site | SEQ ID NO:14 |
| | | | serine. | |
| 3 | H225W, | Full-length, | Mutation of the | SEQ ID NO:15 |
| | S385A | double mutant. | active site | SEQ ID NO:16 |
| | | | histidine and | |
| | | | serine. | |
| 4 | C-terminal | Terminates at | Removing all | SEQ ID NO:17/ |
| | truncation | residue M425. | of the cystein- | SEQ ID NO:18 |
| | M425 | | rich region | |
| | | | (CRR). | |
| 5 | C-terminal | Terminates at | Removes most | SEQ ID NO:19/ |
| | truncation | residue Q453. | of the CRR (all | SEQ ID NO:20 |
| | Q453 | | 18 cysteine | |
| | | | residues are | |
| | | | removed). | |
| 6 | C-terminal | Terminates at | Removes much | SEQ ID NO:21/ |
| | truncation | residue V507. | of the CRR (15 | SEQ ID NO:22 |
| | V507 | | of 18 cysteine | |
| | | | residues are | |
| | | | removed). | |
| 7 | Deletion of | Subtilase | Removes entire | SEQ ID NO:23/ |
| | residues | deletion, | subtilase | SEQ ID NO:24 |
| | L147-M425, | large. | domain. | |
| | inclusive | | | |
| 8 | Deletion of | Subtilase | Removes much | SEQ ID NO:25/ |
| | residues | deletion, | of the sub- | SEQ ID NO:26 |
| | Q218-A392, | small. | tilase domain, | |
| | inclusive | | including | |
| | | | active site | |
| | | | residues. | |

*Residue number corresponds to amino acid residues of SEQ ID NO:9.

Cloning of the Various NARC1 Mutants.

All of the above were cloned as HindIII-XhoI fragments into the following vectors: (1) pEGFP-N2 cut with HindIII and Sal I and (2) pcDNA3.1mycHis(+)C cut with HindIII and XhoI. In these constructs, EGFP and myc & 6XHis are encoded as C-terminal fusion tags. Techniques and protocols for cloning are known in the art, non-limiting examples of which can be found in, e.g., Ausubel et al. (1998) Current Protocols in Molecular Biology (John Wiley & Sons).

Investigation of the NARC1 Autocatalytic Activity.

As indicated above, NARC1 was assigned to the subtilase-like family of polypeptides by bioinformatics. Accordingly, wild type and mutant NARC1 molecules were investigated for autocatalytic properties as follows.

1. Wild-Type NARC1 Expression Reveals Three Polypeptide Products.

The wild-type NARC1-myc/His clone was transiently expressed in COS-7 cells. The cells were then harvested, lysed, and the protein extracted. Polyacrylamide gel electrophoresis was then carried out on the protein extracts, followed by Western blotting. The resulting membranes were probed using both anti-myc- and NARC1-specific antibodies. Routine techniques and protocols were followed. See, e.g., Ausubel et al.(1998), supra.

Western analysis of the wild-type NARC1 transiently expressed as a C-terminal myc fusion in COS cells showed the full-length 74 kD product along with two other products at 71 kD and 60 kD. The lowest molecular weight 60 kD band is the result of normal autoprocessing. This cleavage event maps to approximately the junction between the pro-domain and the enzymatic domain.

2. The NARC1 Point Mutants Demonstrate Reduced or Abolished Autoprocessing.

Analysis of the mutant molecules expressed as a C-terminal myc fusion in COS revealed the following. The 60 kD processing product was reduced upon Western blotting of the site-directed point mutant, H225W, demonstrating reduced autoprocessing. The S385A and double mutant did not produce the 60 kD band, evidencing an absence of autoprocessing for these molecules.

Expression of the EGFP clones in COS-7 cells produced quantitatively similar patterns on Western blots.

3. NARC1 Autoprocessing Appears to be Strictly Intramolecular.

Autoprocessing of NARC1 was investigated as follows. COS-7 cells were co-transfected with the following EGFP and myc plasmids: (A) wild-type NARC1-myc and wild-type NARC1-EGFP; (B) S385A NARC1-myc and S385A NARC1-EGFP; and (C) wild-type NARC1-myc and S385A NARC1-myc. The cells were then harvested, lysed, and the protein extracted. Polyacrylamide gel electrophoresis was then carried out on the protein extracts, followed by Western blotting. The resulting membranes were sequentially probed using first the anti-myc and then the anti-EGFP monoclonal antibodies (Abs). Thus, the experiment allowed sequential detection of the separate NARC1 products encoded from the two co-transfected plasmids.

The results of the Western analysis with the anti-myc and then anti-EGFP monoclonal Abs revealed the following. In the lane representing the wild-type NARC1-myc and S385A NARC1-myc co-transfection, no processing product was detected with the anti-myc Ab. However, a processing product was detected in the same lane probed with anti-GFP.

Those results indicate that wild-type NARC I encoded from the GFP vector cleaves itself, but it leaves the mutant protein, encoded from the myc vector, unprocessed. The absence of intermolecular cleavage in this lane is indicative that the NARC1 autoprocessing is strictly intramolecular.

4. NARC1 Expression in E. coli Exhibits the Same Autoprocessing Activity Observed in Mammalian Cells.

When wild-type NARC1 was expressed in E. coli, it also underwent autoprocessing. The N-terminus of the processed protein was determined by protein microsequencing. Results confirm that the processing product of mammalian-expressed protein (from COS cells) has the same N-terminus.

5. C-Terminal Truncation Mutants Retain Autoprocessing Activity.

As indicated above, a series of C-terminal truncation mutants were generated. See Table 3, rows 4-6. The truncation NARC1-myc/His clones were transiently expressed in COS-7 cells. The cells were then harvested, lysed, the protein extracted, and polyacrylamide gel electrophoresis carried out on the protein extracts. Western analysis was then carried out using both anti-myc and NARC1-specific antibodies. All truncation mutants showed autoprocessing upon Western blotting. The largest of the truncation mutant construct tested (V507 truncation) induced an increased rate of processing.

TABLE 4
Autoprocessing Activity of Wild-Type and Mutant NARC1 Molecules.

| Amino Acid | | | Autoprocessing |
|---|---|---|---|
| Residue Affected* | | Effect | Activity |
| 1 | H225W | Mutation of the active | Reduced processing. |
| | | site histidine. | |
| 2 | S385A | Mutation of the active | Absence of processing |
| | | site serine. | |
| 3 | H225W, S385A | Mutation of active site | Absence of processing |
| | | histidine and serine. | |
| 4 | C-terminal | Removing all of the | Normal processing. |
| | truncation M425 | cysteine-rich region | |
| | | (CRR). | |
| 5 | C-terminal | Removes most of the | Normal processing. |
| | truncation Q453 | CRR (all 18 cysteine | |
| | | residues are removed). | |
| 6 | C-terminal | Removes much of the | Marked increase in |
| | truncation V507 | CRR (15 of 18 cysteine | processing. |
| | | residues are removed). | |
| 7 | Deletion of | Removes entire subtilase | Absence of processing. |
| | residues L147- | domain. | |
| | M425, inclusive | | |
| 8 | Deletion of | Removes much of the | Absence of processing. |
| | residues Q218- | subtilase domain, | |
| | A392, inclusive | including active site | |
| | | residues. | |

*Residue number corresponds to amino acid residues of SEQ ID NO:9

Overexpression Studies of NARC1 Mutants in CGNs.

To investigate NARC1-mediated neuronal cell death, transient expression of the EGFP clones was carried out in cerebellar granule neurons (CGNs). These studies are set forth in detail as follows.

1. The Point Mutants of NARC1 Possess Pro-Apoptotic Activity.

Following transient expression of the wild-type and point-mutant NARC1 constructs in CGNs, the percentage of GFP-positive cells undergoing apoptosis was determined by Laser-Scanning Cytometry. Results demonstrated that wild-type NARC1 is moderately pro-apoptotic, and this effect can be partially rescued by administration of 100 μM BAF. The H225W mutant has essentially a wild-type phenotype, whereas the S385A mutant, as well as the double mutant, has a reduced, but still substantial, pro-apoptotic effect.

2. Effect of Serineprotease Inhibitors Upon Wild-Type NARC1 Activity.

The serine protease inhibitors Aminoethyl-benzene sulfonyl fluoride, an irreversible inhibitor (AEBSF), Aprotinin, a reversible inhibitor, and N-Tosyl-lysine-chloromethylketone (TLCK, an inhibitor known to covalently bind to serine proteases) were investigated to see if any of these reagents can rescue the cell death effects of wild-type NARC1. Caspase 9 transfection was included to test for specificity of any rescue effects.

With a 24-hour exposure, AEBSF demonstrated the most convincing dose-dependent rescue of cell death in NARC1 transfectants, but AEBSF also showed substantial rescue of Caspase 9-induced cell death, indicating an absence of specificity. Aprotinin and TLCK also showed some suggestion of rescue at 24 hours. With a 48-hour exposure, aprotinin and TLCK demonstrated increased effectiveness, while AEBSF appeared toxic.

3. C-Terminal Truncation Mutants Induce Wild-Type Levels of Cell Death in CGNs that is BAF Insensitive.

CGNs were transfected with each of the C-terminal truncation mutants described above. Following transient expression of these constructs, the percentage of GFP positive cells undergoing apoptosis was determined by Laser-Scanning Cytometry. All of the truncation mutants induced wild-type levels of death in CGNs. However, administration of 100 μM BAF did not significantly rescue cell death. Thus, cell death induced by the truncated NARC1 molecules is BAF insensitive.

4. Overexpression Studies of NARC1 in Non-Neuronal Cell Lines Generally Do Not Reveal Cell-Death Promotion Activity.

Wild-type and mutant NARC1 were overexpressed in SY5Y cells (a human neuroblastoma cell line). Nuclear condensation was assessed by Hoechst assay. The results of this study indicate that NARC1 does not promote cell death in this cell line, a result that is typical of non-neuronal cell lines.

However, unlike other cell lines that have been tested in this research effort, Ntera2 cells (human terato-carcinoma, i.e., undifferentiated, cells) show some sensitivity to NARC1 expression. Specifically, Ntera2 cells were transfected with C-terminal EGFP constructs of wild-type NARC1, each of the point-mutant NARC1s, and caspase 9. At 24 hours, nuclear area condensation was assessed for the entire Ntera2 cell population and compared to nuclear area condensation for the transfected cell population. Elevated levels of nuclear condensation in the transfected cells was observed, indicating some sensitivity to NARC1. As compared to neurons, Ntera2 cells appear to be equally sensitive to the S385A mutant and double mutant.

TABLE 5
Cell Death Activity of Wild Type and Mutant NARC1 Molecules.

| Amino Acid | | | Resulting |
|---|---|---|---|
| Residue Affected* | | Effect | Cell Death Activity |
| 1 | H225W | Mutation of the active | Wild type phenotype. |
| | | site histidine. | |
| 2 | S385A | Mutation of the active | Reduced activity. |
| | | site serine. | |
| 3 | H225W, S385A | Mutation of active site | Reduced activity. |
| | | histidine and serine. | |
| 4 | C-terminal | Removing all of the | Induces wild type |
| | truncation M425 | cysteine-rich region | levels of cell death, |
| | | (CRR). | but cell death is BAF |
| | | | insensitive. |
| 5 | C-terminal | Removes most of the | Induces wild type |
| | truncation Q453 | CRR (all 18 cysteine | levels of cell death, |
| | | residues are removed). | but cell death is BAF |
| | | | insensitive. |
| 6 | C-terminal | Removes much of the | Induces wild type |
| | truncation V507 | CRR (15 of 18 cysteine | levels of cell death, |
| | | residues are removed). | but cell death is BAF |
| | | | insensitive. |
| 7 | Deletion of | Removes entire subtilase | Reduced activity. |
| | residues L147- | domain. | |
| | M425, inclusive | | |
| 8 | Deletion of | Removes much of the | Reduced activity. |
| | residues Q218- | subtilase domain, | |
| | A392, inclusive | including active site | |
| | | residues. | |

*Residue number corresponds to amino acid residues of SEQ ID NO:9.

Subcellular Localization of NARC1.

Wild-type NARC1 and the point-mutant NARC1 C-terminal EGFP and myc constructs were transiently expressed and immunofluorescence studies carried out by low resolution microscopy. The wild-type NARC1 protein displayed a peri-nuclear, ER/golgi subcellular localization. The H225W and S385A point mutants did not have demonstrably altered distribution.

Summary.

To review, results of mutational analysis of NARC1 indicate that the H225W point mutation reduces autoprocessing, but has no effect on the cell death phenotype. The S385A point mutation and the H225W, S385A double mutation both eliminate NARC1 autoprocessing and somewhat reduce the pro-apoptotic effect of NARC1. C-terminal truncation mutants have a normal NARC1 pro-apoptotic effect, but the cell death is BAF insensitive (as compared to wild-type NARC1 cell death, which is partially rescued by BAF). In addition, the V507 C-terminal truncation mutant has a markedly increased level of autoprocessing. The subtilase domain deletion mutants behave very similarly to the S385A mutant (slightly reduced pro-apoptotic effect, no autoprocessing).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

That which is claimed:

1. An isolated nucleic acid molecule selected from the group consisting of:
a) a nucleic acid molecule consisting of the nucleotide sequence of SEQ ID NO:10, SEQ ID NO:18, SEQ ID NO:20, or SEQ ID NO:22;
b) a nucleic acid molecule which encodes a polypeptide consisting of the amino acid sequence of SEQ ID NO:9, SEQ ID NO:17, SEQ ID NO:19, or SEQ ID NO:21;
c) a nucleic acid molecule comprising the nucleotide sequence of SEQ ID NO:12, SEQ ID NO:14, SEQ ID NO:16, SEQ ID NO:24, or SEQ ID NO:26;
d) a nucleic acid molecule which encodes a polypeptide comprising the amino acid sequence of SEQ ID NO:11, SEQ ID NO:13, SEQ ID NO:15, SEQ ID NO:23, or SEQ ID NO:25;
e) a nucleic acid molecule comprising a fragment of at least 100 nucleotides of SEQ ID NO:12, SEQ ID NO:14, SEQ ID NO:16, SEQ ID NO:24, or SEQ ID NO:26;
f) a nucleic acid molecule which encodes a fragment of at least 50 contiguous amino acids of SEQ ID NO:11, SEQ ID NO:13, SEQ ID NO:15, SEQ ID NO:23, or SEQ ID NO:25;
g) a nucleic acid molecule comprising a nucleotide sequence having at least 70% sequence identity to the nucleotide sequence of SEQ ID NO:12, SEQ ID NO:14, SEQ ID NO:16, SEQ ID NO:24, or SEQ ID NO:26, wherein the nucleic acid molecule encodes a polypeptide that is incapable of autoprocessing;
h) a nucleic acid molecule consisting of the complement of a), b), c), d), e), f) or g).

2. The isolated nucleic acid molecule of claim 1, wherein the group consists of:
a) a nucleic acid molecule consisting of the nucleotide sequence of SEQ ID NO:10, SEQ ID NO:18, SEQ ID NO:20, or SEQ ID NO:22;
b) a nucleic acid molecule which encodes a polypeptide consisting of the amino acid sequence of SEQ ID NO:9, SEQ ID NO:17, SEQ ID NO:19, or SEQ ID NO:21;
c) a nucleic acid molecule comprising the nucleotide sequence of SEQ ID NO:12, SEQ ID NO:14, SEQ ID NO:16, SEQ ID NO:24, or SEQ ID NO:26;
d) a nucleic acid molecule which encodes a polypeptide comprising the amino acid sequence of SEQ ID NO:11, SEQ ID NO:13, SEQ ID NO:15, SEQ ID NO:23, or SEQ ID NO:25;
e) a nucleic acid molecule consisting of the complement of a), b), c) or d).

3. The nucleic acid molecule of claim 1, further comprising vector nucleic acid sequences.

4. The nucleic acid molecule of claim 1, further comprising nucleic acid sequences encoding a heterologous polypeptide.

5. A host cell which contains the nucleic acid molecule of claim 3.

6. The host cell of claim 5 which is a mammalian host cell.

7. An isolated polypeptide selected from the group consisting of:
a) a polypeptide which is encoded by a nucleic acid molecule consisting of the nucleotide sequence of SEQ ID NO:10, SEQ ID NO:18, or SEQ ID NO:20, SEQ ID NO:22;
b) a polypeptide consisting of the amino acid sequence of SEQ ID NO:9, SEQ ID NO:17, SEQ ID NO:19, or SEQ ID NO:21;
c) a polypeptide which is encoded by a nucleic acid molecule comprising the nucleotide sequence of SEQ ID NO:12, SEQ ID NO:14, SEQ ID NO:16, SEQ ID NO:24, or SEQ ID NO:26;
d) a polypeptide comprising the amino acid sequence of SEQ ID NO:11, SEQ ID NO:13, SEQ ID NO:15, SEQ ID NO:23, or SEQ ID NO:25;
e) a fragment of a polypeptide comprising the amino acid sequence of SEQ ID NO:11, SEQ ID NO:13, SEQ ID NO:15, SEQ ID NO:23, or SEQ ID NO:25, wherein the fragment comprises at least 50 contiguous amino acids of SEQ ID NO:11, SEQ ID NO:13, SEQ ID NO:15, SEQ ID NO:23, or SEQ ID NO:25;
f) a polypeptide having at least 70% sequence identity to the amino acid sequence SEQ ID NO:1, SEQ ID NO:13, SEQ ID NO:15, SEQ ID NO:23, or SEQ ID NO:25, wherein the polypeptide lacks autoprocessing activity.

8. The isolated polypeptide of claim 7, wherein the group consists of:
a) a polypeptide which is encoded by a nucleic acid molecule consisting of the nucleotide sequence of SEQ ID NO:10, SEQ ID NO:18, SEQ ID NO:20, or SEQ ID NO:22;
b) a polypeptide consisting of the amino acid sequence of SEQ ID NO:9, SEQ ID NO:17, SEQ ID NO:19, SEQ ID NO:21;
c) a polypeptide which is encoded by a nucleic acid comprising the nucleotide sequence of SEQ ID NO:12, SEQ ID NO:14, SEQ ID NO:16, SEQ ID NO:24, or SEQ ID NO:26; and
d) a polypeptide comprising the amino acid sequence of SEQ ID NO:11, SEQ ID NO:13, SEQ ID NO:15, SEQ ID NO:23, or SEQ ID NO:25.

9. The polypeptide of claim 7, further comprising heterologous amino acid sequences.

10. An antibody which selectively binds to a polypeptide of claim 7.

11. A method for producing a polypeptide comprising culturing the host cell of claim 5 under conditions in which the nucleic acid molecule is expressed.

12. A method for detecting the presence of a polypeptide of claim 7 in a sample, comprising the steps of:
a) contacting the sample with a compound which selectively binds to a polypeptide of claim 7; and
b) determining whether the compound binds to the polypeptide in the sample.

13. The method of claim 12, wherein the compound which binds to the polypeptide is an antibody.

14. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

15. The method of claim 14, wherein the sample comprises mRNA molecules and is contacted with a nucleic acid probe.

16. A method for identifying a compound which binds to a polypeptide of claim 7, comprising the steps of:
a) contacting a polypeptide of claim 7, or a cell expressing a polypeptide of claim 7, with a test compound; and
b) determining whether the polypeptide binds to the test compound.

17. The method of claim 16, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for programmed cell death activity.

18. A method for modulating the activity of a polypeptide of claim 7, comprising contacting a polypeptide of claim 7, or a cell expressing a polypeptide of claim 7, with a compound which binds to the polypeptide in a sufficient concentration to modulate the activity of the polypeptide.

19. A method for identifying a compound which modulates the activity of a polypeptide of claim 7, comprising:
a) contacting a polypeptide of claim 7 with a test compound; and
b) determining the effect of the test compound on the activity of the polypeptide to thereby identify a compound that modulates the activity of the polypeptide.

20. The method of claim 19, wherein the compound is an inhibitor of programmed cell death activity.
